(19) United States
(12) Patent Application Publication
(10) Pub. No.: US 2022/0161019 A1
(43) Pub. Date: May 26, 2022

(54) PURGELESS MECHANICAL CIRCULATORY SUPPORT SYSTEM WITH MAGNETIC DRIVE

(71) Applicant: Kardion GmbH, Stuttgart (DE)

(72) Inventors: Marvin Mitze, Stuttgart (DE); Hans Christof, Unterensingen (DE); Vladimir Popov, Stuttgart (DE); Martin Schwarz, Wangen (DE); Leon Wenning, Stuttgart (DE); Johannes Bette, Balingen (DE); Attila Fabiunke, Karlsruhe (DE); Sina Gerlach, Esslingen (DE); Johannes Stigloher, Stuttgart (DE); Julian Görries, Stuttgart (DE); Jan Schöfer, Stuttgart (DE); Valentin Rex, Aachen (DE); Johannes Berner, Stuttgart (DE); Bernhard Ehni, Aichelberg (DE); Johannes Ferch, Esslingen (DE); Hans-Baldung Luley, Stuttgart (DE); Tom Döhring, Esslingen (DE); Jens Burghaus, Stuttgart (DE); Inga Schellenberg, Stuttgart (DE); Hardy Baumbach, Stuttgart (DE); Annika Bach, Leutenbach (DE); Ingo Stotz, Ditzingen (DE); Julian Kassel, Boblingen (DE); Armin Schuelke, Aidlingen (DE); Stefan Henneck, Leonberg (DE); David Minzenmay, Stuttgart (DE); Thomas Alexander Schlebusch, Renningen (DE); Tobias Schmid, Stuttgart (DE); Tjalf Pirk, Stuttgart (DE); Martina Budde, Karlsruhe (DE); Ricardo Ehrenpfordt, Kornta-Munchingen (DE); Marc Schmid, Stuttgart (DE); Ahmad Mansour, Weil der Stadt (DE); Niko Baeuerle, Bad Liebenzell (DE); Ralf Strasswiemer, Ravenstein (DE); Uwe Vollmer, Sindelfingen (DE); Manuel Gaertner, Burgstetten (DE); Fabian Eiberger, Gerlingen (DE); Tobias Baechle, Stuttgart (DE); Karin Schneider, Herrenberg (DE); Peter Wassermann, Weil der Stadt (DE)

(21) Appl. No.: 17/455,662

(22) Filed: Nov. 18, 2021

Related U.S. Application Data
(60) Provisional application No. 63/116,616, filed on Nov. 20, 2020.
(60) Provisional application No. 63/116,686, filed on Nov. 20, 2020.

(51) Int. Cl. A61M 60/237; A61M 60/148; A61M 25/09

(52) U.S. Cl. CPC A61M 60/237; A61M 60/148; A61M 2025/09083; A61M 2025/09175; A61M 2210/125; A61M 25/09041

(57) ABSTRACT

Disclosed is a minimally invasive miniaturized percutaneous mechanical circulatory support system. The system may be placed across the aortic valve via a single femoral arterial access point. The system includes a low profile axial rotary blood pump carried by the distal end of a catheter. The system can be percutaneously inserted through the femoral artery and positioned across the aortic valve into the left ventricle. The device actively unloads the left ventricle by pumping blood from the left ventricle into the ascending aorta and systemic circulation. A magnetic drive and encased motor housing allows for purgeless operation for extended periods of time to treat various ailments, for example more than six hours as acute therapy for cardiogenic shock.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims priority to U.S. Provisional Application No. 63/116,616, titled MECHANICAL LEFT VENTRICULAR SUPPORT SYSTEM FOR CARDIOGENIC SHOCK and filed on Nov. 20, 2020, and to U.S. Provisional Application No. 63/116,686, titled MECHANICAL CIRCULATORY SUPPORT SYSTEM FOR HIGH RISK CORONARY INTERVENTIONS and filed on Nov. 20, 2020, the entire contents of each of which is incorporated by reference herein in its entirety for all purposes and forms a part of this specification.

### BACKGROUND

Cardiogenic shock (CS) is a common cause of mortality, and management remains challenging despite advances in therapeutic options. CS is caused by severe impairment of myocardial performance that results in diminished cardiac output, end-organ hypoperfusion, and hypoxia. Clinically this presents as hypotension refractory to volume resuscitation with features of end-organ hypoperfusion requiring immediate pharmacological or mechanical intervention. Acute myocardial infarction (MI) accounts for over about 80% of patients in CS.

Miniature, catheter based intracardiac blood pumps are used as an acute therapy for CS patients. However, current generation pumps include performance deficiencies such as, for example, inadequate blood flow, the requirement for ongoing motor purging within the pump, undesirably high hemolysis, and inadequate sensing of hemodynamic parameters. Thus, there remains a need for a circulatory support system that overcomes these and other drawbacks, and which may be specifically configured to treat CS patients.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for mechanical circulatory support systems.

The following disclosure describes non-limiting examples of some embodiments. For instance, other embodiments of the disclosed systems and methods may or may not include the features described herein. Moreover, disclosed advantages and benefits can apply only to certain embodiments and should not be used to limit the disclosure.

A minimally invasive miniaturized percutaneous mechanical left ventricular support system is provided, optimized for treatment of patients experiencing cardiogenic shock. The system includes a low profile (e.g., 18 Fr to 19 Fr) mechanical circulatory support (MCS) device which includes an axial rotary blood pump and an elongate inlet tube, carried by the distal end of a nine French catheter. The system can be positioned to span the MCS device across the aortic valve into the left ventricle, where it actively unloads the left ventricle by pumping blood from the left ventricle into the ascending aorta and systemic circulation, and may provide flow rates of up to about 6 L per minute at 60 mmHg. In some embodiments, flow rates between 0.6 L per minute and 6 L per minute may be provided.

Intravascular access may be achieved using an 8 to 14 Fr (e.g., 8 to 10.5 Fr) introducer sheath, expandable to accommodate an 18 to 19 French MCS device. Access may be via percutaneous transfemoral puncture, or axillary access via a surgical cut down.

The introducer sheath can be part of an introducer kit that may also include a guidewire, a dilator, an insertion tool, and a guidewire aid.

The motor is completely sealed by encapsulation within a motor housing, having a magnetic coupling to allow the motor to drive the impeller without the need for a shaft to leave the housing. The magnetic coupling includes a cylindrical driving magnet array positioned within the motor housing, concentrically positioned within a cylindrical driven magnet array located outside of the motor housing and mechanically coupled to the impeller. The impeller rotates with respect to the motor housing about a pivot jewel bearing. The magnetic coupling is flushed by a constant blood flow through flushing holes on proximal and distal ends of the magnetic coupling. The sealed motor enables elimination of a purging process necessary for certain competitive devices.

Migration may optionally be inhibited by an intravascular anchor carried by the catheter shaft, which provides anchoring in the aorta. The anchor may include a plurality of radially outwardly expandable struts, carried by the catheter shaft, configured to contact the wall of the aorta and anchor the shaft against migration while allowing perfusion through the anchor struts.

Migration may optionally be inhibited by a locking mechanism that engages the catheter shaft in a fixed position with an introducer sheath that is held to an arteriotomy with sutures, thus holding the catheter shaft still relative to the endovascular access pathway.

Onboard sensors enable real time actual measurement of any of a variety of parameters of interest, such as aortic pressure, left ventricular pressure (including LVEDP) temperature and blood flow velocity or others depending upon the desired clinical performance. Sensors may be included on a distal end of the device, such as distal end of an inlet tube on a distal side of the blood outflow port. Additional sensors may be provided on the proximal end of the elongate body, such as proximal to the blood outflow ports.

Specific sensors may include at least a first MEMS pressure and temperature sensor for direct measurement of absolute left ventricular pressure. Sensors also enable extraction of important physiological parameters such as LVEDP. Ultrasound transducers may be provided, for direct measurement of blood flow volume through the pump or optionally around the pump. Ultrasound transducer surfaces may be curved and configured for increased focus and high sensitivity. A second MEMS pressure and temperature sensor may be provided on the proximal end of the inlet tube, such as to enable direct measurement of absolute aortic pressure and allow for differential pressure measurement. Alternatively or additionally, other forms of sensors may be used to assess flow rate such as laser doppler, thermal or electrical impedance sensors

Flexible electrical conductors may extend along the length of the inlet tube for connecting distal and proximal sensors into an integrated system. The flexible conductors may be in the form of a flexible PCB, which extends axially in a spiral around the inlet tube, in between the proximal and distal sensors. Multi conductor cable bundles extend proximally through the elongate, flexible tubular body, to connectors at a proximal manifold, for releasable connection to an external electronic control unit.

A mechanical ventricular support system for cardiogenic shock may be provided. The system may include an elongate flexible catheter shaft, having a proximal end and a distal end, a mechanical circulatory support carried by the distal end of the shaft, the mechanical circulatory support including a mechanical circulatory support housing, a motor, rotationally fixed with respect to a drive magnet array, an impeller, rotationally fixed with respect to a driven magnet array, and a sealed motor housing, inside of the mechanical circulatory support housing, and encasing the motor and the drive magnet array. The system may include a removable guidewire guide tube. The guide tube may enter a first guidewire port on a distal end of the housing, exit the housing via a second guidewire port on a side wall of the housing distal to the impeller, reenter the housing via a third guidewire port on a proximal side of the impeller, and extend proximally into the catheter shaft. The system may include at least one inlet port and at least one outlet port on the housing separated by a flexible section of the housing. The distance between the inlet port and outlet port may be at least about 60 mm and no longer than 100 mm, preferably 70 mm. The system may include a first pressure sensor proximate the inlet port. The system may include a second pressure sensor on a proximal side of the outlet port. The system may include a visual indicium on the catheter shaft, within the range of from about 50 mm to about 150 mm from the distal end of the catheter shaft (or beginning of the pump). The motor may be positioned distal to the third guidewire port. The system may include an ultrasound transducer proximate the inlet port. The system may include a guidewire aid removably carried by the mechanical circulatory support. The guidewire aid can include a tubular body having a distally facing opening and an inside diameter that increases in the distal direction to the opening. The guidewire aid may include a guidewire guide tube attached to the body. The guidewire guide tube can include a split line for splitting the guide tube so that the guide tube can be peeled away from a guidewire extending through the tube. The flexible section of the housing may include a flexible slotted tube covered by an outer polymeric sleeve.

A mechanical ventricular support system for high-risk coronary interventions may be provided. The system may include a ventricular support catheter, including a mechanical circulatory support carried by an elongate flexible catheter shaft, a sealed motor and an impeller inside the mechanical circulatory support and rotationally coupled together by a magnetic bearing, an insertion tool having a tubular body and configured to axially movably receive the mechanical circulatory support, and an access sheath, having a tubular body and configured to axially movably receive the insertion tool. The access sheath may include an access sheath hub having a first lock for engaging the insertion tool. The access sheath hub may include a second lock for engaging the catheter shaft.

A controller configured to drive a motor of a mechanical circulatory support system may be provided, wherein the controller does not include a purging component. The purging component can include a cassette or a port. In some embodiments, the system does not require purging.

A controller configured to drive a motor of a mechanical circulatory support system having a housing for mounting electronic components and a handle disposed on a top portion of the housing may be provided. The controller can include a visual alarm element wrapped around the handle on the top portion of the housing. In some embodiments, the housing may not include more than one control element. The control element can be a rotary dial. The control element may be positioned on a first end of the housing. The controller may include a cable management system, said cable management system positioned on a second end opposite the first end. The controller may include a rotating securing attachment on a rear side of the housing.

Various other example aspects and embodiments are shown and described throughout this disclosure. For example, various particular example embodiments are further described herein in the Detailed Description in the section “Example Embodiments.”

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a cross sectional rendering of a heart showing an embodiment of a mechanical circulatory support (MCS) device of the present invention carried by a catheter and positioned across an aortic valve via a femoral artery access.

FIG. 2 schematically illustrates the access pathway for a MCS system.

FIG. 3 is a side elevational view of an embodiment of a MCS system in accordance with the present disclosure.

FIG. 4 shows the system of FIG. 3, with the introducer sheath removed and including an insertion tool and a guidewire loading aid.

FIG. 5 shows an introducer kit having a sheath and dilator.

FIG. 6 shows an embodiment of a placement guidewire.

FIG. 7 is a partial perspective view of a distal pump region of an embodiment of a MCS device.

FIGS. 8A and 8B are a side elevational view of a distal region of an embodiment of a MCS device and an enlarged view of a distal portion of a guidewire for the MCS device, respectively.

FIG. 9A is a cross sectional view through the impeller and magnetic coupling region of the MCS device of FIG. 7.

FIG. 9B shows a cross sectional view through an embodiment of the rotor bearing system at a location where the first permanent magnet, which is arranged in the housing, and the second permanent magnet, which is arranged in the rotor, overlap.

FIG. 9C shows an alternative embodiment to FIG. 9B.

FIGS. 9D and 9E each show a rotor bearing and magnetic coupling system according to further embodiments.

FIG. 10 is a perspective view of an embodiment of a MCS device.

FIG. 11 is a perspective view of another embodiment of a MCS device.

FIG. 12 is a perspective view of another embodiment of a MCS device.

FIG. 13 is a perspective view of another embodiment of a MCS device.

FIG. 14 is a perspective view of an embodiment of an impeller housing of a MCS device.

FIG. 15 is a side view of an embodiment of a pump of a MCS device.

FIG. 16 is a side view of another embodiment of a pump of a MCS device.

FIG. 17 is a side view of another embodiment of a pump of a MCS device.

FIG. 18 is a side, schematic view of an embodiment of a magnetic radial rotary coupling of a MCS device.

FIG. 19 is cross-sectional view of an embodiment of a sensor head unit of a MCS device.

FIG. 20 is cross-sectional view of another embodiment of a sensor head unit of a MCS device.

FIGS. 21A and 21B are a perspective view and a cross-sectional view, respectively, of an interface between the distal end of the catheter shaft and a proximal end of a MCS device.

FIG. 22 is a perspective, exploded view of a MCS device and an enlarged view showing an example sensor arrangement.

FIGS. 23A and 23B are a schematic view and a perspective view, respectively, of an ultrasound transducer.

FIG. 24 is a schematic diagram of an embodiment of a method of taking Doppler measurements of a fluid flowing through a MCS device.

FIG. 25 is a schematic view showing a cross-section of a distal end of an embodiment of a MCS device with an ultrasound transducer for taking Doppler measurements.

FIG. 26 is a schematic, side view of an alternative embodiment of a MCS system with an ultrasound transducer for taking Doppler measurements.

FIG. 27 is a schematic diagram of another embodiment of a method of taking Doppler measurement of a fluid flowing through a MCS device.

FIG. 28 is a schematic diagram showing an embodiment of a MCS device with temperature sensors positioned in a patient.

FIG. 29 is a schematic diagram showing an embodiment of a MCS system with a monitoring device.

FIG. 30A is a front elevational view of a MCS controller.

FIG. 30B is a rear perspective view of the controller of FIG. 30A.

FIG. 31 illustrates a block diagram of an electronic system that can be housed inside the controller of FIGS. 30A and 30B.

FIG. 32 illustrates an exploded view with components of the electronic system of FIG. 31 inside the controller.

FIG. 33 illustrates a side perspective view of the MCS controller of FIG. 30A.

FIG. 34A illustrates a graph showing pressure difference between aortic pressure and left ventricular pressure.

FIG. 34B illustrates a graph showing applied current for a constant velocity.

FIG. 35 illustrates an example user interface for displaying parameters.

FIG. 36A illustrates an example user interface in a configuration mode.

FIG. 36B illustrates an example user interface in an operating mode.

FIG. 37 illustrates an embodiment of an electronic control element.

FIGS. 38A, 38B, 38C, and 38D illustrate a process for determining LVEDP.

### DETAILED DESCRIPTION

The following detailed description is directed to certain specific embodiments of a mechanical circulatory support (MCS) system and method, and related features. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

The mechanical circulatory support (MCS) device of the present invention is a temporary (generally no more than about 6 days) support system for enhancing cardiac output in cardiogenic shock patients such as caused by acute ST elevation myocardial infarction. It is placed across the aortic valve typically via transvascular access, and pumps blood from the left ventricle to the ascending aorta.

One implementation of the system includes an 18 to 19 Fr axial rotary blood pump and inlet tube assembly mounted on a catheter such as a catheter no larger than 10.5 Fr. When in place, the ventricular support pump can be driven by the ventricular support controller to provide at least about 4 or 5 and up to about 6.0 liters/minute of partial left ventricular support, at about 60 mm Hg pressure differential. No system purging is needed due to the encapsulated motor and magnetic bearing design.

An expandable sheath allows 8 to 14 Fr (e.g., 8 to 10.5) initial access size for easy insertion and closing, expandable to allow introduction of at least about a 14 Fr and preferably an 18 to 19 Fr device. Access may be accomplished via transfemoral, transaxillary, transaortal or transapical approach.

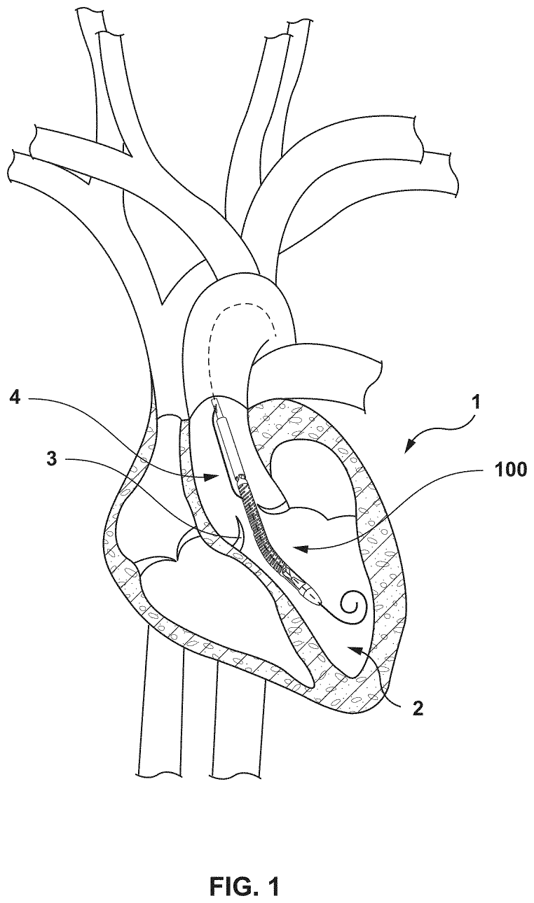

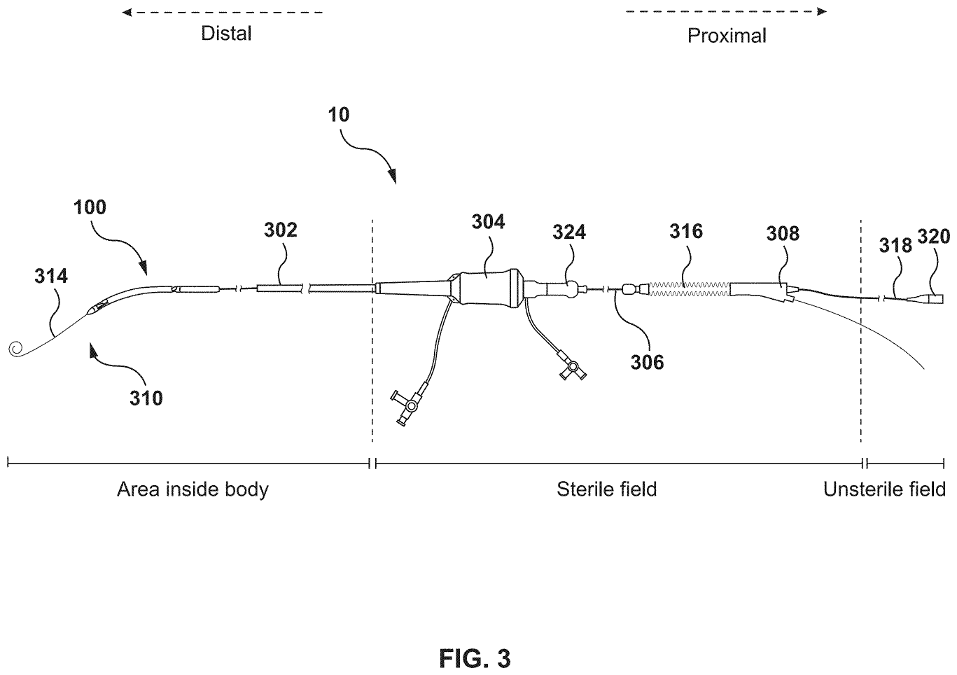

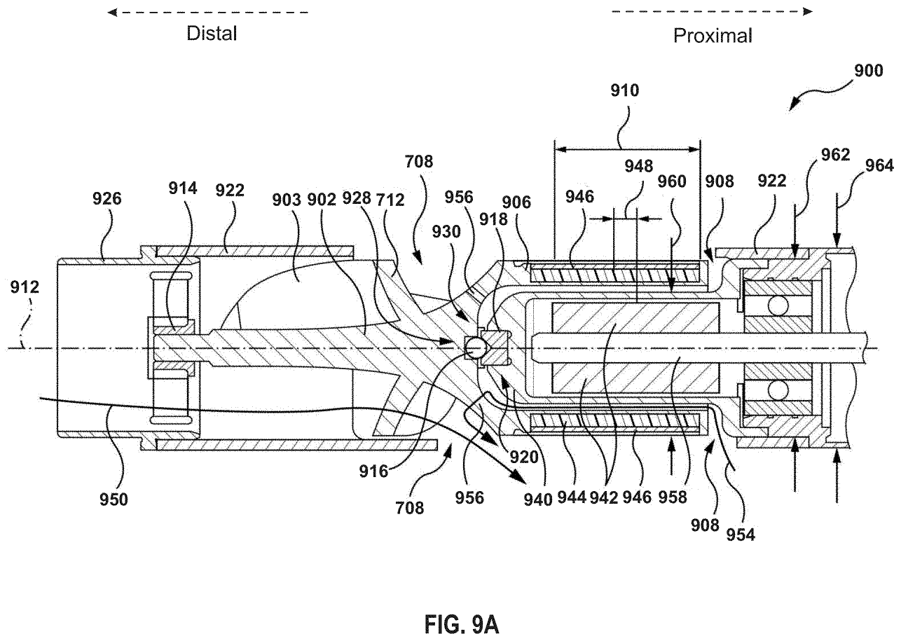

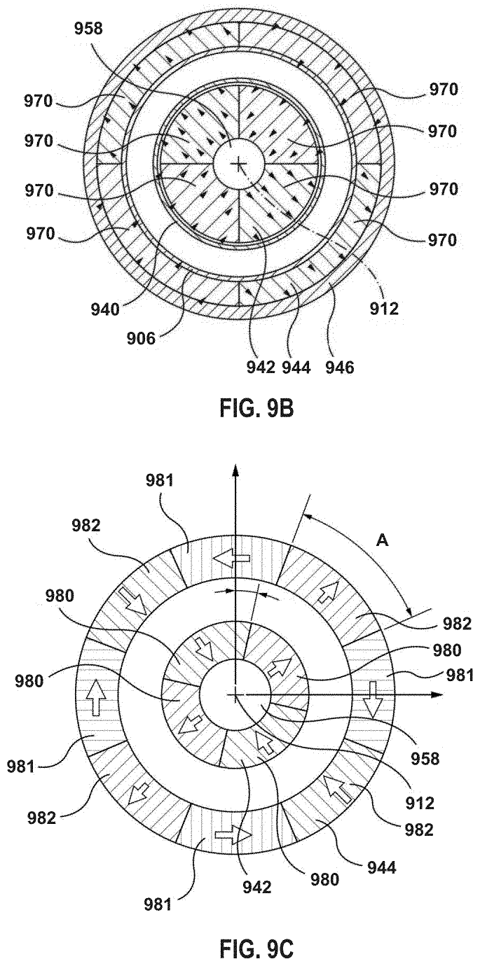

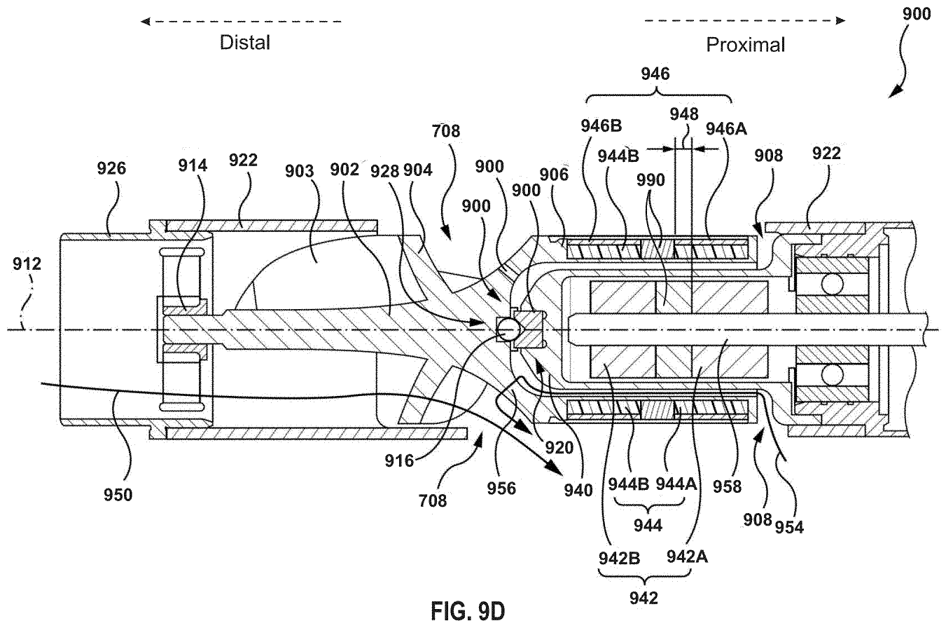

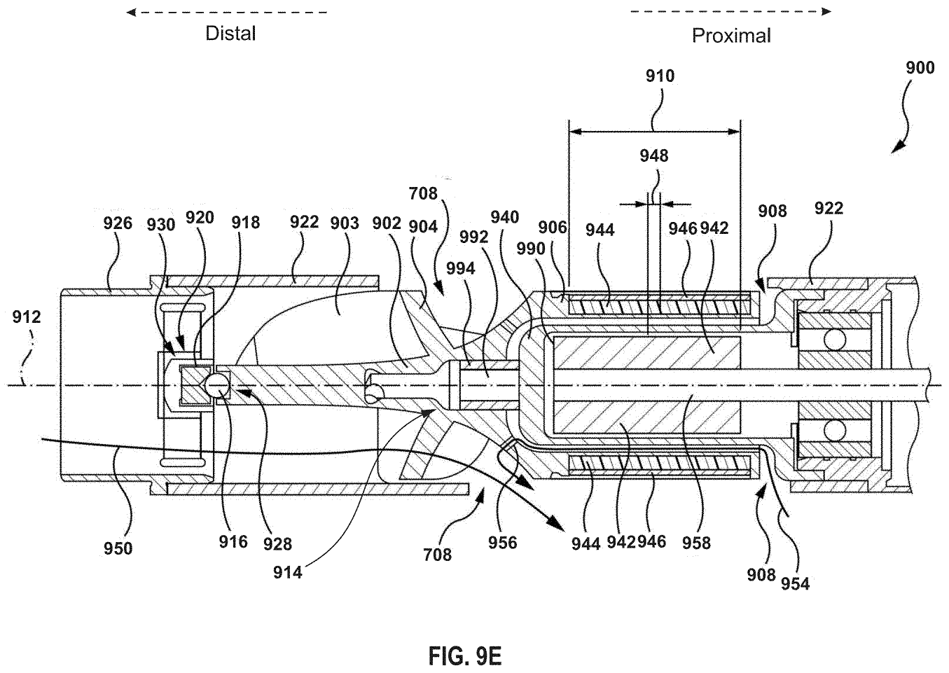

FIG. 1 shows an example embodiment of a MCS device 100 mounted on a distal end of a catheter (for example, a catheter 300 shown in FIG. 3). In the illustrated embodiment, an inlet tube portion of the MCS device 100 extends across the aortic valve 3 of a heart 1. An impeller (for example, see FIG. 9A) of the MCS 100 is located at least partially at the outflow section 4 (in the ascending aorta) of the inlet tube drawing blood from the left ventricle 2 of the heart 1 and ejecting it into the ascending aorta. The MCS device 100 can includes a motor, which can be mounted directly proximal to the impeller in an encapsulated housing eliminating the need to purge the motor prior to or during use. The MCS device 100 can actively unload blood from the left ventricle 2 by pumping blood from the ventricle 2 and ejecting the pumped blood into the ascending aorta 4 and systemic circulation.

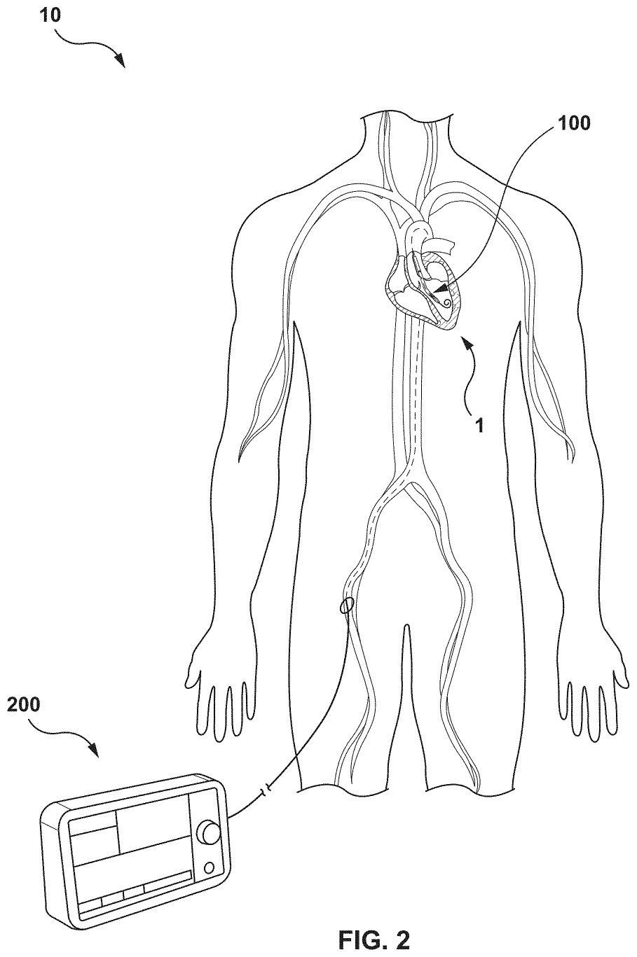

FIG. 2 shows an embodiment of a MCS system including the MCS device 100 in connection with a MCS controller 200. When in place, the MCS device 100 can be driven by the MCS controller 200 to provide between about 0.4 l/min and about 6.0 l/min of, for example, partial left ventricular support. In some embodiments, the MCS device 100 can be driven by the MCS controller 200 to provide between about 0.6 l/min and about 6.0 l/min of, for example, partial left ventricular support. A range between about 0.61/min and about 6.0 l/min may allow for 10 equidistant flow levels, for example.

In general, the overall MCS device 100 can include a series of related subsystems and accessories, including one or more of the following. The MCS device 100 may include a catheter shaft with an inlet tube, an impeller, a motor, and sensors and associated housings, and a proximal hub, an insertion tool, a proximal cable, an infection shield, a guidewire guide tube, and/or a guidewire aid. The MCS device 100 may be provided sterile. The MCS device 100 may contain the electrical cables and a guidewire lumen for over-the-wire insertion. The proximal hub may contain guidewire outlet with a valve to maintain hemostasis and connect the ventricular support shaft to the proximal cable, where the proximal cable connects the MCS device 100 to the MCS controller 200. The proximal cable may be about 3.5 m (approx. 177 inch) in length and extend from a sterile field to a non-sterile field where the MCS controller 200 is located. An MCS device insertion tool can be a part of the MCS device 100 to facilitate the insertion of a pump of the MCS device 100 into an introducer sheath and to protect an inlet tube and hemostasis valves from potential damage or interference when passing through the introducer sheath. A peel-away guidewire aid may be pre-mounted on the MCS device 100 to facilitate the insertion of a guidewire, for example an 0.018″ placement guidewire, into the inlet tube and into the MCS shaft. A 3 m 0.018″ placement guidewire may be used, having a soft coiled pre-shaped tip for atraumatic wire placement into the left ventricle. The guidewire may be provided sterile. An introducer sheath that is expandable between a low profile in a range of 8 to 14 Fr (e.g., 8 to 10.5 Fr) to a larger profile in a range of 14 to 21 Fr (e.g., 14 to 19 Fr) with a usable length of at least about 250 mm or 275 mm may be used. The introducer sheath may maintain access into the femoral artery and provide hemostasis for the 0.035″ guidewire, the 5 to 6 Fr diagnostic catheters, the 0.018″ placement guidewire, and the insertion tool. The housing of the introducer sheath may accommodate the MCS insertion tool. The introducer sheath may be provided sterile. An introducer dilator compatible with the introducer sheath may be used to facilitate atraumatic insertion of the introducer sheath into the femoral artery. The introducer dilator may be provided sterile. An MCS controller 200 may be used, which drives and operates the MCS device 100, observes its performance and condition as well as providing error and status information. The powered controller may be designed to support at least about 12 hours of continuous operation and contains a basic interface to indicate and adjust the level of support provided to the patient. Moreover, the MCS controller 200 may provide an optical and/or audible alarm notification in case the MCS device 100 detects an error during operation. The MCS controller 200 may be provided non-sterile and be contained in an enclosure designed for cleaning and re-use outside of the sterile field. The controller enclosure may contain a socket into which the extension cable is removably plugged.

Referring to FIG. 3, there is illustrated an embodiment of the MCS system 10 in accordance with one aspect of the present intention, subcomponents of which will be described in greater detail below. For reference, the “distal” and “proximal” directions are indicated by arrows in a number of figures. “Distal” and “proximal” as used herein have their usual and customary meaning, and include, without limitation, a direction more distant from an entry point of the patient's body as measured along the delivery path, and a direction less distant from an entry point of the patient's body as measured along the delivery path, respectively.

The MCS system 10 includes an introducer sheath 302 having a proximal introducer hub 304 with a central lumen for axially movably receiving a MCS shaft 306 and being expandable to axially movably receive the MCS device 100. The MCS shaft 306 extends between a proximal hub 308 and a distal end 310. The hub 308 may be provided with an integrated microcontroller for device identification and tracking of the running time which could be used to prevent overuse to avoid excessive wear or other technical malfunction. The microcontroller or memory device could disable the device, for example to prevent using a used device. They could communicate with the controller, which could display information about the device or messages about its usage. An atraumatic cannula tip with radiopaque material allows the implantation/explantation to be visible under fluoroscopy.

The MCS device 100 can include a tubular housing. The tubular housing of the MCS device 100 is used broadly herein and may include any component of the MCS device 100 or component in a pump region of the MCS device 100, such as an inlet tube, a distal endpiece, a motor housing, other connecting tubular structures, and/or a proximal back end of the motor housing. The MCS device 100, for example the tubular housing, may be carried by a distal region of the MCS shaft 306. The MCS device 100 may be provided with at least one central lumen for axially movably receiving a guidewire 314. The proximal hub 308 is additionally provided with an infection shield 316. A proximal cable 318 extends between the proximal hub 308 and a connector 320 for releasable connection to a control system typically outside of the sterile field, to drive the MCS device 100 and communicate with sensors in the MCS device 100.

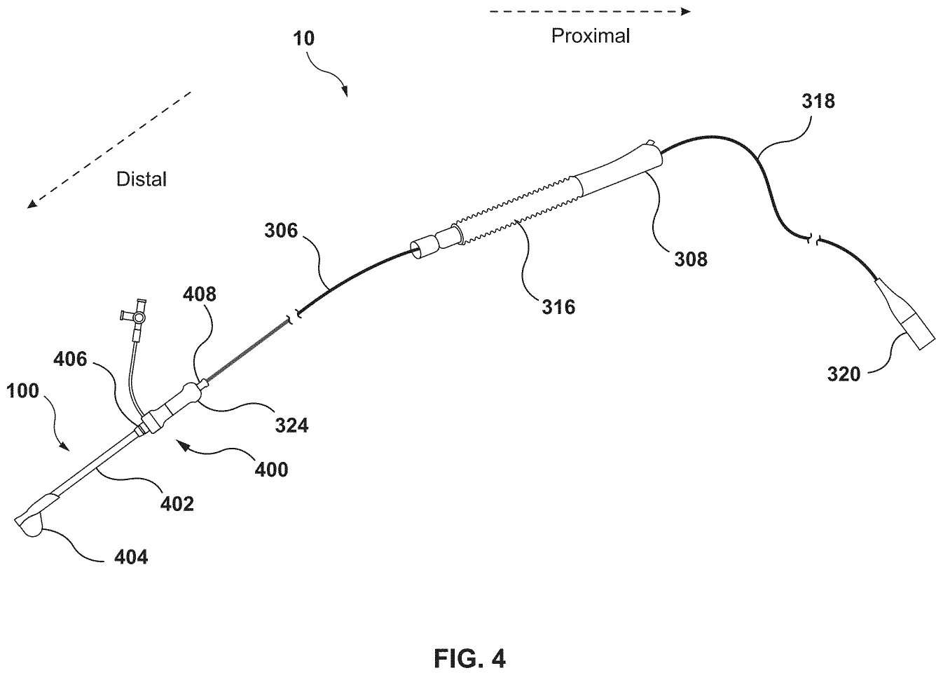

Referring to FIG. 4, the MCS system 10 may include an insertion tool 400, having an elongate tubular body 402 having a length able to contain the MCS device 100 (not shown here), for example within the range of from about 85 mm to about 160 mm (e.g., about 114 mm) and an inside diameter able to slidable contain the MCS device 100, for example within the range of from about 6 mm to about 6.5 mm, extending distally from a proximal hub 406. The tubular body 402 may include a central lumen adapted to axially movably receive the shaft 306 and the MCS device 100 there through, and sufficient collapse resistance to maintain patency when passed through the hemostatic valves of the introducer sheath. As illustrated in FIG. 4, the MCS device 100 can be positioned within the tubular body 402, such as to facilitate passage of the MCS device 100 through the hemostatic valve(s) on the proximal end of an introducer hub 304. In some embodiments, a marker 722 (see FIG. 7) can be provided on the shaft 306 spaced proximally from the distal tip 704 (see FIG. 7) such that as long as the marker 722 is visible on the proximal side of the hub 324, the clinician knows that the MCS device 100 is within the tubular body 402.

The hub 324 may be provided with a first engagement structure 406 for engaging a complimentary second engagement structure on the introducer sheath 302 (not shown) to lock the insertion tool into the introducer sheath 302. The hub 324 may also be provided with a locking mechanism 408 for clamping onto the shaft 306 to prevent the shaft 306 from sliding proximally or distally through the insertion tool 400 once the MCS device 100 has been positioned at the desired location in the heart. The hub 324 may additionally be provided with a hemostasis valve to seal around the shaft 306 and also accommodate passage of a pump having the larger diameter. In some embodiments, the MCS device 100 as packaged is prepositioned within the insertion tool 400 and the guidewire aid 404 is pre-loaded within the MCS device 100 and the shaft 306, as illustrated in FIG. 4.

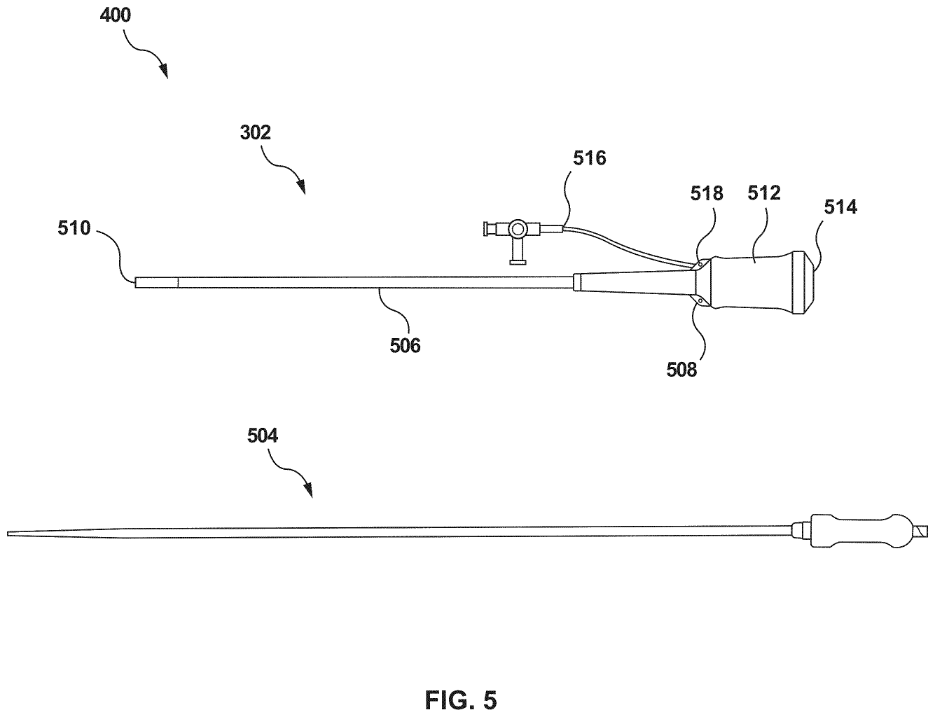

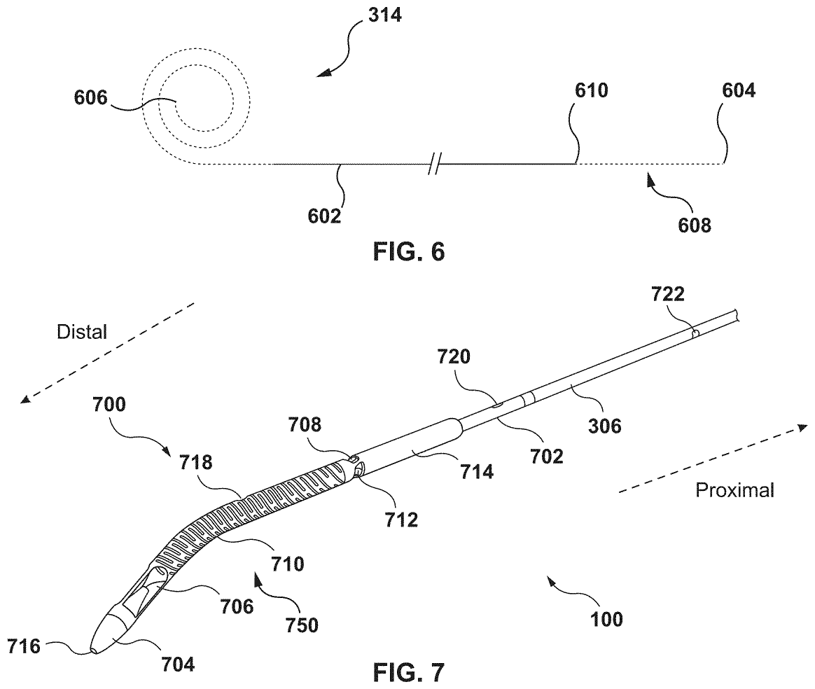

Referring to FIGS. 5 and 6, the insertion tool 400 may include a guidewire 314, an introducer sheath 302, a dilator 504, and a guidewire aid 404 (shown in FIG. 4). The guidewire 314 may include an elongate flexible body 602 extending between a proximal end 604 and a distal end 606. A distal zone of the body 602 may be pre-shaped into a J tip or a pigtail, as illustrated in FIG. 6, to provide an atraumatic distal tip. A proximal zone 608 may facilitate threading into and through the MCS device 100 and may extend between the proximal end 604 and a transition 610. The proximal zone 608 may have an axial length within the range of from about 100 mm to about 500 mm (e.g., about 300 mm).

The introducer tool 400 may comprise a sheath 302 and/or a dilator 504. The sheath 302 may comprise an elongate tubular body 506, extending between a proximal end 508 and a distal end 510. The tubular body 506 may terminate proximally in a proximal hub 512. Optionally, the tubular body 506 may be expandable or may be peeled apart. The proximal hub 512 may include a proximal end port 514 in communication with a central lumen extending throughout the length of the tubular body 506 and out through a distal opening, configured for axially removably receiving the elongate dilator 504. The proximal hub 512 may additionally be provided with a side port 516, and at least one and optionally two or more attachment features such as an eye 518 to facilitate, for example, suturing to the patient, and at least one and optionally a plurality of hemostasis valves for providing a seal around a variety of introduced components such as a standard 0.035″ guidewire, a 5 Fr or 6 Fr diagnostic catheter, an 0.018″ placement guidewire 314, and the insertion tool 400.

FIG. 7 illustrates additional details of a distal pump region 700 of the MCS device 100, showing the MCS device 100 and a distal portion of the catheter shaft 306. The distal pump region 700 extends between a bend relief 702 at the distal end of shaft 306 and a distal tip 704 (or nose piece). The MCS device 100 may include a tubular housing 750 which may include an inlet tube (or inlet cannula) 710, a distal tip (or nose piece) 704, and/or a motor housing 714. The tubular housing 750 may include one or more pump inlets 706 and/or outlets 708, which may be part of the inlet tube 710, or part of other structures such as an intermediate structure joining a proximal end of the inlet tube 710 to the motor housing 714. A guidewire guide aid, as further described herein, may extend into and out of various components of the system, such as the tubular housing 350 and/or the catheter shaft 306 of the MCS device 100.

A pump inlet (or inlet windows) 706 comprising one or more windows or openings is in fluid communication with the pump outlet (or outlet windows) 708 comprising one or more windows or openings by way of a flow path extending axially through the inlet tube (or inlet cannula) 710. The pump inlet (or inlet windows) 706 may be positioned at about the transition between the inlet tube 710 and the proximal end of distal tip 704. The pump inlet 706 may be generally within about 5 cm, 3 cm, or less distance from the distal port 716.

In some embodiments, the distal tip 704 is radiopaque. For example, the distal tip 704 may be made from a polymer containing a radiopacifier such as barium sulfate, bismuth, tungsten, iodine. In some embodiments, an entirety of the MCS device 100 may be radiopaque. In some embodiments, a radiopaque marker is positioned on the inlet tube between the pump outlet 708 and the guidewire port 718 to indicate the current position of the aortic valve.

The inlet tube 710 may comprise a highly flexible slotted (e.g., laser cut) metal (e.g., Nitinol) tube having a polymeric (e.g., Polyurethane) tubular layer to isolate the flow path. The inlet 710 tube may have an axial length within the range of from about 60 mm and about 100 mm, and in one implementation is about 67.5 mm. The outside diameter may be within the range of from about 5 mm to about 6.5 mm, and in one implementation is about 5.5 mm. The connections between the inlet tube 710 and the distal tip 704 and to the motor (or motor housing 714) may be secured such as through the use of laser welding, adhesives, threaded or other interference fit engagement structures, or may be via press fit.

An impeller 712 may be positioned in the flow path between the pump inlet 706 and pump outlet 708 (see for example FIG. 9A). In the illustrated embodiment, the impeller 712 is positioned adjacent to the pump outlet 708. As is discussed further below, the impeller 712 is rotationally driven by a motor contained within motor housing 714. In some embodiments, the motor driving the impeller 712 is positioned on the proximal side of the impeller 712.

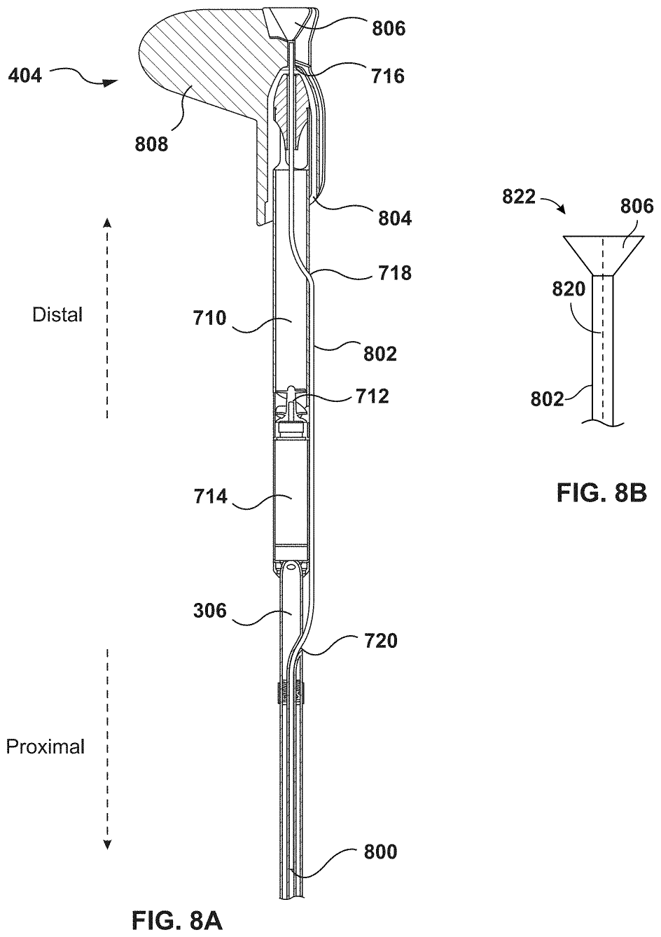

FIGS. 8A and 8B are a side, cross-sectional view and a detail view respectively of the distal pump region 700 of an embodiment of the MCS device 100 showing an embodiment of the guidewire aid 404. The MCS device 100 can be provided in either a rapid exchange configuration or over the wire configuration. In a rapid exchange configuration, a first guidewire port 716 (such as a distal-facing opening) on a distal face of the distal tip 704 may be in communication, via a first guidewire lumen through the distal tip 704 and at least a portion of the flow path in the inlet tube 710, with a second guidewire port 718 extending, for example, through a side wall of the inlet tube 710, and distal to the impeller 712. This allows the guidewire 314 to exit the inlet tube 710 of the distal pump region 700 at the second guidewire port 718 and extend proximally along the outside of the catheter or the shaft 306 from the second guidewire port 718.

In an over the wire configuration, the guidewire 314 may extend proximally throughout the length of the catheter or the shaft 306 through a guidewire lumen therein. In the over the wire embodiment illustrated in FIG. 7, however, the guidewire 314 exits the inlet tube 710 via second guidewire port 718, extends proximally across the outside of the impeller 712 and motor housing 714, and reenters the shaft 306 via a third guidewire port 720. The third guidewire port 720 may be located proximal to the motor, and, in the illustrated embodiment, is located (or formed) on the bend relief 702. Third guidewire port 720 may be in communication with a guidewire lumen of the shaft 306 which extends proximally throughout the length of the shaft 306 and exits at a proximal guidewire port carried by the proximal hub 308.

As shown in FIG. 8A, the pump may be provided assembled with the removable guidewire aid 404 having a guidewire guide tube 802 which tracks the intended path of the guidewire from the first guidewire port 716, proximally through the distal piece (or nose piece) 704 and back outside of the inlet tube 710 via the second guidewire port 718 and back into the shaft 306 via the third guidewire port 720. In the illustrated embodiment, the guidewire guide tube 802 may extend proximally within the shaft 306 to a proximal end 800, in communication with, or within the guidewire lumen, which extends to the proximal hub 308 (see FIG. 4). The proximal end 800 may be positioned within about 5 mm or 10 mm of the distal end of the shaft 306, or may extend into the lumen of the shaft 306 for at least about 10 mm or 20 mm, such as within the range of from about 10 mm to about 50 mm. In some embodiments, the third port 720 may be located within a proximal end of the tubular housing, such as the motor housing or backend, or in any other components of the device at a location that is proximal to the impeller.

The guidewire aid 404 may have a funnel 806. The funnel 806 may be located at a distal end of the guide tube 802 and provided pre-positioned at a distal end of the inlet tube, for example at the distal tip or nose piece 704. The funnel 806 may increase in width in the distal direction, from a narrow proximal end in communication with the guide tube 802, to a wider distal opening at a distal end of the funnel 806. The funnel 806 may be conical, frustoconical, pyramidal, segmented, or other shapes. A proximal end of the funnel 806 may be attached to a distal end of the guidewire guide tube 802. The proximal end 604 of the guidewire 314 (see FIG. 6) may be inserted into the funnel 806, passing through the first (distal) guidewire port 716 and guided along the intended path by tracking inside of the guidewire guide tube 802. The guidewire guide tube 802 may then be removed by sliding the guide tube 802 distally out of the distal tip 704 and peeling it apart longitudinally, leaving the guidewire 314 in place.

The guidewire aid 404 may have a pull tab 808. In some embodiments, a distal end of the guidewire guide tube 802 is attached to the pull tab 808 of the guidewire aid 404. The pull tab 808 may be a structure capable of being gripped by a human hand, for example with a lateral, planar extension as shown. The guidewire aid 404, for example, the pull tab 808, the guide tube 802 and/or the funnel 806, may be provided with a tearable line 820, as more clearly shown in FIG. 8B. The tearable line 820 may be an axially extending split line. The tearable line 820 may include a weakening, a slot, or a perforated linear region. Removal of the guidewire aid 404 may be accomplished such as by grasping the pull tab 808 and pulling out the guidewire tube 802 and/or funnel 806 and removing them from the guidewire 314 as they split or peel away along the split line 820, such as shown in the detailed inset 822 of FIG. 8B.

The guidewire aid 404 may include a proximal opening 804 configured to slip over and removably receive the distal end of the MCS device 100, in particular the nose piece 704 and fragile struts that define the inlet openings 706 (see FIG. 7). A guidewire guide tube 802 having a lumen therethrough may be positioned within the proximal opening 804 and aligned to pass through the guidewire port 716 of the distal tip 704. The proximal opening 804 may further be configured to slip over and removably receive a distal end of tubular body 402 of an insertion tool 400 as shown in FIG. 4. The MCS device 100 may be dimensioned so that an annular space defined between the outer surface of the MCS device 100 such as the inlet tube 710, the motor housing 714, the bend relief (or strain relief) 702, and the inner surface of the tubular body 402 of the insertion tool 400, may removably receive the guidewire guide tube 802 therein, when the MCS device 100, guidewire aid 404 and insertion tool 400 are assembled together.

In some embodiments, the lumen of the guidewire guide tube 802 may be in communication with a distal flared funnel opening 806 which gets larger in cross-section in the distal direction. The guidewire aid 404 may be provided assembled on the MCS device 100 with the guidewire guide tube 802 pre-loaded along a guidewire path, for example into the MCS device 100 through the guidewire port 716, through a portion of the fluid path within the inlet tube 710, out of the MCS device 100 through the port 718, along the exterior of the MCS device 100 and back into the shaft 306 through the port 720. This helps a user guide the proximal end of a guidewire 314 into the funnel 806 through the guidewire path and into the guidewire lumen of the MCS shaft 306. A pull tab 808 may be provided on the guidewire aid 404 to facilitate grasping and removing the guidewire aid 404, including the guidewire guide tube 802, following loading of the guidewire 314. The guidewire aid 404 may have a longitudinal slit or tear line 820, for example along the funnel 806, proximal opening 804 and guidewire guide tube 802, to facilitate removal of the guidewire aid 404 from the MCS device 100 and guidewire 314.

In one implementation, the distal end of the guidewire guide tube 802 is attached to the guidewire aid 404. The guide tube 802 may be provided with an axially extending split line such as a weakening, slot or perforated tearable line. Removal of the guide tube 802 may be accomplished such as by grasping the pull tab 808 and pulling out the guide tube 802 as it splits along the split line to release the guidewire 314. The inside surface of guide tube 802 may be provided with a lubricious coating, such as PTFE.

The guidewire aid 404 features described herein may be used with a variety of different MCS systems and/or pump devices. The guidewire aid 404 may be used for guidewire paths that enter and exit a pump housing, as described, or that do not exit a housing. The guidewire aid 404 is described herein as being used with an MCS system configured for temporary operation for high-risk PCI procedures. The system may include rotating impeller with a radial shaft seal and a motor rotating the impeller via a shaft extending through the seal. The guidewire aid 404 may be used with a variety of different devices. The guidewire aid 404 may also be used with a pump having a magnetic drive, where the motor rotates a first magnet within a sealed motor housing that magnetically communicates with a second magnet of the impeller that is external to the sealed housing to rotate the impeller. Thus, the guidewire aid 38 is not limited to use with only the particular pump embodiments described herein.

FIG. 9A illustrates an example embodiment of a pump 900 (for example, a rotor bearing system) that can provide contactless torque transfer and include a radial and axial motor mount. The rotor bearing system 900 can serve as a pump for MCS devices or systems described herein.

The pump 900 has a housing 940 that encapsulates a motor, drive shaft, and drive magnet array hermetically sealed from the surrounding environment. Within the housing 940, a first magnet array 942 (or drive magnet array) may be seated on a shaft 958 that can be driven by a motor (not shown). The first magnet array 942 may rotate about a first axis 912.

The housing 940 may have a first cylindrical portion having a first outer diameter 964 (for example, in a range of 5 to 7 mm, preferably 6 mm) that radially encompasses the motor (not shown), a second cylindrical portion having a second outer diameter 962 that is less than the first outer diameter (e.g., less than the first outer diameter by a range of 0.3 to 1 mm, preferably by 0.5 mm), and a third cylindrical portion having a third outer diameter 960 that is less than the second outer diameter (e.g., less than the second outer diameter by 1.7 to 2.3 mm, preferably by 2.0 mm).

The second cylindrical portion with the second outer diameter 962 may securely mate with an inlet tube housing 922, wherein the second cylindrical portion and the inlet tube housing 922 may be sized so the outer diameter of the inlet tube housing 922 is flush with the outer circumference of the first cylindrical portion having the first outer diameter 964 (for example, the thickness of the inlet tube housing 922 may be equal to the difference between the first outer diameter and second outer diameter divided by 2). The third outer diameter 960 of the housing 940 may be, for example, in a range of 3.2 to 3.8 mm, preferably 3.5 mm.

Additionally, the pump 900 may comprise the impeller 712 for conveying a liquid. The impeller 712 may include the second magnet array 944 (or driven magnet array) in the form of, for example, a hollow cylinder mounted to rotate about the first axis 912. The second magnet array 944 may positioned within a hollow, cylindrical jacket 906 such that the second magnet 944 is arranged in a form of a hollow cylinder. The jacket 906 may include a back-iron 946 positioned around (or covering) a radial exterior of the second magnet array 944.

In some embodiments, the first magnet array 942 may have an outer diameter of 3 mm, a magnet height of 1 mm, and a length of 3.2 mm (e.g., in a range of 3 to 4.2 mm). The second magnet array 944 may have an outer diameter of 5.3 mm (e.g., in a range of 5 to 5.3 mm), a magnet height of 0.6 mm (e.g., in a range of 0.5 to 0.6 mm), and a length of 3.2 mm (e.g., in a range of 3 to 4.2 mm). The stagger 948 between the first magnet array 942 and the second magnet array 944 may be 1 mm (e.g., in a range of 0.1 to 1.2 mm). The jacket 906 of the impeller 712 may have an outer diameter of 5.3 mm (e.g., less than the second outer diameter 962 by a range of 0.1 to 0.4, preferably 0.2 mm) and a length of 15 mm.

The impeller 712 may convert the mechanical power transferred by the shaft 958 into hydraulic power to convey a blood flow against a blood pressure. Additionally, the impeller 712 may comprise a tapered portion 902 that integrated with the jacket 906. The tapered portion 902 may be conical in shape. The outer circumference of the base surface of the tapered portion 902 may be connected with the outer circumferential surface of the jacket 906.

The first magnet array 942 (or drive magnet array) and the second magnet array 944 (or driven magnet array) at least partially axially overlap in an overlap area 910. As shown in FIG. 9A, the first magnet array 942 may be axially staggered in relation to the second magnet array 944. In the embodiment illustrated in FIG. 9A, the centers of the first magnet array 942 and the second magnet array 944 are marked by vertical lines, wherein the axial stagger 948 is drawn between these two vertical lines.

Due to the axial stagger 948, the second magnet array 944 may experience a force directed to the right in FIG. 9A, so that a ball 916 of the impeller 712 is pushed onto a cone 918 arranged in the housing 940, and a first bearing 920 and a third bearing 928, which in this case form a combined axial and radial bearing 930, are held in contact. Alternatively, the ball 916 may be a part of the housing 940 and the cone 918 may be a part of the impeller 712. In some embodiments, the ball 916 may rotate in the cone 918, so that both radial and also axial forces can be absorbed and the axial and radial bearing is achieved. The combined axial and radial bearing 930 may be a solid body bearing. In some embodiments, the ball 916 is arranged in the tapered portion 902.

The ball 916 for example, may have a diameter in a range of 0.5 mm to 0.9 mm, preferably 0.7 mm, and the cone 918 may have a diameter of 1 mm, a height of 0.8 mm, and a cone angle within a range of 70° to 90°, preferably 80°. The combined bearing 930 may provide relative axial positioning of the impeller 712, the housing 940, and/or the shaft 958 to each other and may absorb an axial force caused by the arrangement (or relative positions) of the first magnet array 942 and the second magnet array 944. Moreover, the axial force on the pump 900 may be adjusted, so that the exerted force settings can be optimized.

A portion of the housing 940 that encapsulates the first magnet array 942, may at least in part be radially surrounded by jacket 906 in the form of a hollow cylinder attached to the impeller 712. A channel 908 in the form of a hollow cylinder may be formed between the housing 940 and the jacket 906 of the impeller 712, through which a liquid (for example, blood) can flow. The impeller 712 may include one or more bores or perforations 956. In some embodiments, the bores 956 are formed in the tapered portion (or conical portion) 902 of the impeller 712, or in a transition portion between the tapered portion 902 and the jacket 906. The bores 956 may be in fluid communication with the channel 908 such that, for example, fluid (for example, blood) can flow into the area between the housing 904 and the jacket 906 via the channel 908 and exit via the bores 956. In some embodiments, when the impeller 712 spins, liquid (for example, blood) is centrifugally expelled from the bores 956 and liquid is pulled into the channel 908 to replace the expelled liquid in a continuous flow. Purging flow 954 indicates the direction of flow of the liquid through the channel 908 and the bores 956. Pump flow 950 indicates the direction of flow of liquid transferred by the vanes 903 of the impeller 912.

During use, a build-up can occur, for example, in an area between the jacket 906 and the housing 940 shown in FIG. 9A, and reduce pumping efficiency of the pump 900. For example, the build-up can reduce the amount of torque transferred between the housing 940 and the impeller 712 and therefore reduce the amount of blood being pumped through the pump outlet 708. For example, the purging flow 954 described herein can prevent or mitigate such build-up caused by flow of blood during use of the pump 900, and allow the pump 900 to operate purgeless (that is, without having to purge).

A second bearing 914 may be as a radial, hydrodynamic, and blood-lubricated plain bearing. The second bearing 914 may be arranged on the end (for example, a distal end) of the tapered portion 902 of the impeller 712 facing away from the housing 940. The second bearing 914 may absorb radial forces applied to the impeller 712 and may position the axis of rotation of the impeller 712 and the second magnet array 944 (or driven magnet array) in alignment with the axis of rotation 912 of the shaft 958 or the first magnet array 942. In the illustrated embodiment (see FIG. 9A), the second bearing 914 may be arranged between the impeller 712 and an insert 926, which may be fastened, in particular clamped in or pressed in, in a ring-shaped, distal end of the second housing 922, which is in turn fastened onto the housing 940. The second housing 922 may form an exterior skin or cover of the pump 900. The second housing 922, which may be referred as an impeller housing, may include one or more outlet windows 708. The insert 926 may be a bearing star that may be firmly attached (for example, glued, welded, or friction fitted) to the second housing 922. The insert 926 may have an outer diameter of 6 mm (e.g., in a range of 5 to 7 mm) and a length of 3 mm (e.g., in a range of 2 to 5 mm). The second housing 922 may have an outer diameter of 6 mm (e.g., in a range of 5 to 7 mm), a length of 18 mm (e.g., in a range of 15 to 21 mm), and a wall thickness of 0.25 mm (e.g., in a range of 0.15 to 0.5 mm).

In some embodiments, the insert 926 and second housing 922 may be manufactured as a single piece, which may have a consistent inner diameter. In this arrangement, an extended inlet cannula may be connected to the combined insert 926 and second housing 922 for example by laser welding.

The second bearing 914 may have a diameter of 1 mm (e.g., in a range of 0.75 to 1.5 mm) and a length of 1 mm (e.g., in a range of 0.75 to 2 mm).

The axial stagger 948 between the first magnet array 942 (or the drive magnet array) and the second magnet array 944 (or the driven magnet array) may generate a defined axial force on the impeller 712 in the proximal direction (that is, from left to right in the exemplary embodiment in FIG. 9A). This force generated by the axial stagger 948 may be opposed by a hydraulic force applied on the impeller 712 during operation in the distal direction (that is, from right to left in the exemplary embodiment in FIG. 9A). This hydraulic force applied on the impeller 712 may be in the opposite direction of the pump flow 950 generated by the spinning vanes 903 of the impeller 712.

In some embodiments, the axial force originating from the coupling of the first magnet array 942 and the second magnet array 944 may be larger than the maximum expected hydraulic force, which ensures that the impeller 712 is at all times held in a defined axial position. By ensuring that the axial force is not too much larger than the maximum expected hydraulic force, the combined axial and radial bearing 930 may not be unnecessarily overloaded, and friction and wear as well as reduction of torque transmitted to the rotor can be minimized. The amount of the axial force may be varied by adjusting one or more of the dimensions (for example, length, thickness, outer diameter) of one or more of the first magnet array 944, the second magnet array 942, the axial stagger 948, and the segment angle, A (if in a Halbach configuration such as the one shown in FIG. 9C).

FIG. 9B shows a cross-section view of an overlapping area (or region) 910 of the pump 900 where the first magnet array 942 and the second magnet array 944 axially overlap. The first magnet array 942 may be seated on the shaft 958 driven by the motor (not shown), where the shaft 958 may rotate about the axis 912. In some embodiments, the shaft 958 may also function as a back-iron. The second magnet array 944 may be mounted such that it can rotate about the axis 912. In the illustrated embodiment, both the first magnet array 942 and also the second magnet array 944 each has two pole pairs, that is to say respectively four poles 970 that are each radially magnetized, which is indicated by small arrows. Alternatively, both the first magnet array 942 and the second magnet array 944 may each have one pole pair or at least one pole pair (for example, two pole pairs, three pole pairs, four pole pairs).

FIG. 9C shows an alternative embodiment of an arrangement of the first magnet array 942 and the second magnet array 944. The inner ring shown in FIG. 9C represents the first magnet array 942 seated on the shaft 958. The shaft 958 may function as a back-iron. The first magnet array 942 may include two pole pairs (or four poles 980) that may be each radially magnetized, indicated by arrows shown in FIG. 9C. The outer ring shown in FIG. 9C represents the second magnet array 944 arranged in a Halbach array. The second magnet array 944 may not need a back-iron. The second magnet array 944 may include four tangentially magnetized magnet ring segments 981 between the four radial segments 982, which can guide the magnetic field generated around the second magnet array 944. For example, this arrangement may efficiently prevent stray magnetic fields outside of the coupling (that is, between the first magnet array 942 and the second magnet array 944) and may increase the efficiency of the magnetic coupling between the first magnet array 942 and the second magnet array 944 (in comparison to the configuration shown in FIG. 9B). In some embodiments, change of the segment angle, A, can change the amount of axial force between the first magnet array 942 and the second magnet array 944.

In some embodiments, the segment angle α is 45° and the pump 900 has an outer diameter (for example, the first outer diameter 964 shown in FIG. 9A) of 6.2 mm. The outer diameter (for example, the first outer diameter 964) of the pump 900 can limit the sizes of the first magnet array 942 and the second magnet array 944. In some embodiments, the inner and outer diameter of the first magnet array 942 are 1.0 mm and 3.0 mm, respectively. In some embodiments, the inner and outer diameter of the second magnet array are 4.1 mm and 5.3 mm, respectively. It is contemplated that the pump 900 can have the outer diameter greater than or less than 6.2 mm, which can result in smaller, larger, or the same sizes or dimensions (for example, the inner diameter and the outer diameter) for the first magnet array 942 and the second magnet array 944.

As discussed herein, the length the first and the second magnet arrays 942, 944, and the stagger 948 may be modified to adjust, for example, the amount axial force and torque generated by the first magnet array 942 and the second magnet arrays 944.

The length of the magnets (the first magnet array 942 and the second magnet arrays 944) can affect movement of the pump 900 of the MCS device 100. In some embodiments, the sum of the magnet length (that is, the length of the first magnet array 942 or the second magnet arrays 944) and stagger 948 may be about 4.2 mm to allow the pump 900 to traverse vascular pathway during endovascular delivery to the heart. In some embodiments, the length of the first magnet array 942 and the second magnet array 944 is about 3.2 mm and the stagger 948 is about 1.0 mm. It is contemplated that the length of the magnets (the first magnet array 942 and the second magnet arrays 944) can be greater than or less than 3.2 mm and the stagger 948 can be less than or greater than 1.0 mm. Because the forces applied to the impeller 712 and coupling between the first magnet array 942 and the second magnet arrays 944 are a function of overall device diameter, inlet tube length, impeller design, maximum impeller speed or blood flow rate, and other features or dimensions that affect hydraulic force, bearing frictional losses, and eddy current losses, the sizes and dimensions of various components or parts described herein may differ with devices having different dimensions or features compared to the ones tested.

FIG. 9D shows another embodiment of the pump 900. In the illustrated embodiment, the first magnet array 942, the second magnet array 944, and the back-iron 946 are each split into two axial segments. In some embodiments, the first magnet array 942, the second magnet array 944, and the back-iron 946 may be split into more than two segments (e.g., three segments, four segments, and the like).

The first magnet array 942 may include segments 942A, 942B. The second magnet array 944 may include segments 944A, 944B. The back-iron 946 may include segments 946A, 946B. The segments 942A, 944A, 946A may be arranged on the motor side (for example, distal or away from the vanes 903 of the impeller 712), and the segments 942B, 944B, 946B may be arranged on the side facing the impeller 712 (for example, proximal to the vanes 903 of the impeller 712).

A spacer 990 may be placed between the segments 942A, 942B of the first magnet array 942, between the segments 944A, 944B of the second magnet array 944, and between the segments 946A, 946B of the back-iron 946. The spacer 990 may be a hollow cylinder mounted on the shaft 958 and positioned between the segments 942A, 942B of the first magnet array 942.

The segmentation of the first magnet array 942, the second magnet array 944, and the back-iron 946 may, in combination with the stagger 150, increase the axial magnetic force between the first magnet array 942 and the second magnet array 944. Additionally, the segmentation may reduce the transferable torque between the first magnet array 942 and the second magnet array 944. Segmenting the magnet arrays 942, 944 and the back-iron 946 may be helpful in situations where the axial magnetic force between the first magnet array 942 and the second magnet array 944 is insufficient to reliably compensate the flow force.

FIG. 9E illustrates another embodiment of the pump 900. In the illustrated embodiment, the second bearing 914 is replaced with the first bearing 920 and the third bearing 928. Additionally, the axial stagger 948 between the first magnet array 942 and the second magnet array 944 points in the opposite direction as in the embodiment in FIG. 9A. For example, in the embodiment illustrated in FIG. 9A, the center of the first magnet array 942 is further away from, for example, the tapered portion 902 of the impeller 712 than the center of the second magnet array 944. In contrast, in the embodiment illustrated in FIG. 9E, the center of the second magnet array 944 is further away from, for example, the tapered portion 902 of the impeller 712 than the center of the first magnet array 942. In the embodiment illustrated in FIG. 9E, the axial stagger 948 may be 1 mm. It is contemplated that the axial stagger 948 can be less than or greater than 1 mm for the embodiment illustrated in FIG. 9E.

The first magnet array 942 and the second magnet array 944 may at least partially axially overlap in the overlap area 910. The first magnet array 942 may be arranged axially staggered in relation to the second magnet array 944. The centers of the first magnet array 942 and the second magnet array 944 are marked by vertical lines, wherein the axial stagger 948 represents the distance between the two vertical lines.

In contrast to the embodiment illustrated in FIG. 9A, the first magnet array 942 is axially staggered in relation to the second magnet array 944 in the direction of the impeller 712 as seen from the housing 940. This may result in a defined axial force between the first magnet array 942 and the second magnet array 944 in the embodiment illustrated in FIG. 9E acting or applied on the impeller 712 in a distal direction (that is, in the direction from the housing 940 towards the impeller 712 along the axis 912). As discussed herein, a hydraulic force may act or apply on the impeller 712 in the same direction (that is, in the direction from the housing 940 towards the impeller 712 along the axis 912).

Advantageously, in the configuration illustrated in FIG. 9E, both the magnetic and the hydraulic axial force are applied on the impeller 712 in the same direction (for example, upstream against the pump flow 950). As such, both the magnetic and the hydraulic axial force press the impeller 712 into the combined axial and radial bearing 930.

The combined axial and radial bearing 930 may be arranged on the end (for example, a distal end) of the tapered portion 902 of the impeller 712 facing away from housing 940. The combined bearing 930 may be arranged between the impeller 712 and the insert 926, which may be fastened to (or clamped into) a ring-shaped end of the second housing 922, which is in turn fastened to the housing 940. The ball 916 may be arranged on the end (for example, a distal end) of the tapered portion 902 of the impeller 712 and pressed onto a cone 918 arranged on or attached to the insert 926.

As discussed herein, the second bearing 914 may include a radial, hydrodynamic plain bearing. The second bearing 914 may absorb radial forces and position the axis of rotation of the second magnet array 944. In the embodiment illustrated in FIG. 9E, the second bearing 914 may be arranged between the housing 940 and the impeller 712. In contrast to the embodiment illustrated in FIG. 9A, the housing 940 of the embodiment illustrated in FIG. 9E may include a cylindrical pin 992 behind a wall 990 facing the impeller 712. The cylindrical pin 992 may be aligned along the axis 912 of the shaft 958 and may extend towards the impeller 712. The pin 992 may be surrounded by a bearing shell 994 to form a radial plain bearing of the second bearing 914.

The pump 900 of the MCS device 100 may include the housing 940, which may house the first magnet array 942. The housing 940 and the first magnet array 942 may rotate about the axis 912. The impeller 712 may include the second magnet array 944. The second magnet array 944 may be in the form of a hollow cylinder and may rotate about an axis of rotation (this axis of rotation may be aligned with the axis 912 shown in FIGS. 9A, 9B, and 9E). As described herein, the first magnet array 942 and the second magnet array 944 may partially overlap, such that the first magnet array 942 is staggered in relation to the second magnet array 944. The overlap area 910 of the pump 900 includes an overlap (for example, axial overlap) between the first magnet array 942 and the second magnet array 944, where a portion of the housing 940 is positioned between the first magnet array 942 and the second magnet array 944. The first bearing 920 may provide axial positioning of the impeller 712 and the housing 940 relative to each other and may absorb axial force (for example, magnetic axial force) that results from the arrangement of the first magnet array 942 and the second magnet array 944. The second bearing 914 and the third bearing 928 may be arranged to absorb radial forces and to position or align an axis of rotation of the second magnet array 944 with respect to the axis 912.

Additional details regarding the embodiments of the motor 900, and other components and/or features such as the impeller 714, magnet arrays 942, 944, bearings and the like shown in FIGS. 9A-9E are described in PCT WO2019229223, filed May 30, 2019, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, which is hereby incorporated by reference in its entirety.

In some embodiments, the impeller 712 may include a medical grade titanium. This enables computational fluid dynamics (CFD) optimized impeller designs that minimize or reduce shear stress and thereby reduce damage to the blood cells (for example, hemolysis). Additionally, the impeller 712 may include a non-constant slope, which can further increase its efficiency. Electro polishing of the surface of the impeller 712 may decrease the surface roughness and therefore minimize the impact on hemolysis.

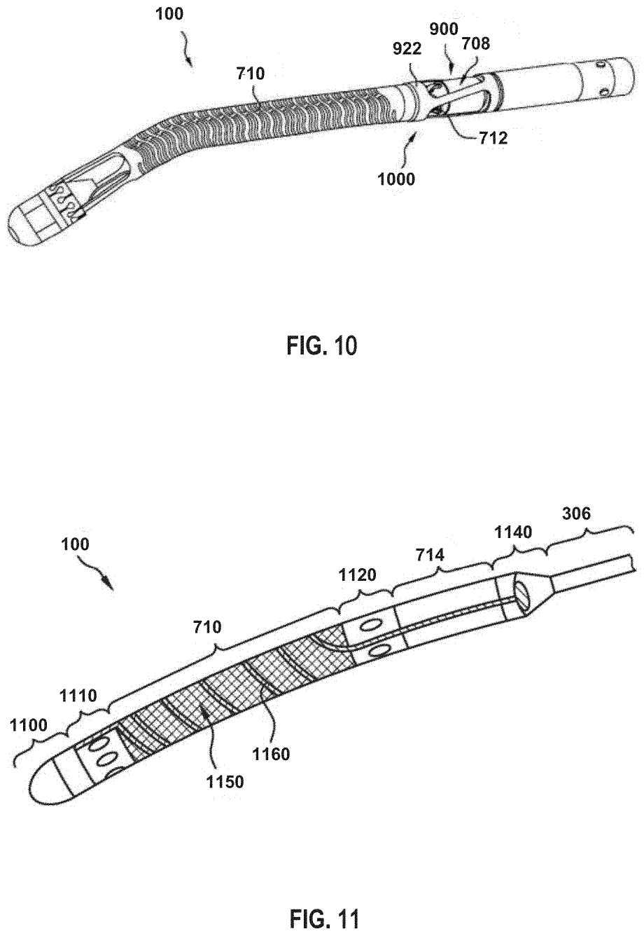

FIG. 10 shows another embodiment of the MCS device 100 having another embodiment of the impeller housing 922.

The MCS device 100 may be arranged in a minimally invasive manner through a transfemoral or transaortic catheter in an aorta and/or at least partially in a ventricle. As described herein, the MCS device 100 may include the pump 900 for facilitate blood flow in the heard of a patient. A maximum external diameter of the MCS device 100 shown in FIG. 10 may be less than ten millimeters (for example, less than or equal to 7 mm, less than or equal to 5 mm). The pump 900 may have an axial design including the impeller 712 (see FIG. 9) against which axial flow occurs. The axial design of the pump 900 can allow the MCS device 100 having the external diameter of less than 10 mm.

During the operation of the MCS device 100, blood may flow through an inlet tube 710 and be expelled through outlet openings 708 formed on the circumference of an impeller housing 922 of the pump 900 and flow into, for example, the aorta. The impeller 712 may be completely enclosed in a cylindrical, first section by the impeller housing 922 without the outlet windows or openings 708 and is followed in a second section of the impeller housing 922 with the outlet openings 708. A transition between these two sections is characterized by proximal edges 1000 of the outlet openings 708.

Additional details regarding the embodiment of FIG. 10 are described in PCT Publication No. WO2019229214, filed May 30, 2019, titled PUMP HOUSING DEVICE, METHOD FOR PRODUCING A PUMP HOUSING DEVICE, AND PUMP HAVING A PUMP HOUSING DEVICE, which is hereby incorporated by reference in its entirety.

FIG. 11 shows another embodiment of the MCS device 100. The MCS device 100 may include a cylindrical, elongated structure with a substantially constant outer diameter and rounded, tapering ends for easy placement by means of a catheter in a blood vessel, for example the left ventricle or the aorta.

The MCS device 100 can include the inlet tube 710 (see FIG. 7) arranged between a sensor head unit 1100, a motor housing 714, an end unit 1140, and a connection cable or the shaft 306 of the MCS device 100. The inlet tube 710 may be connected to the sensor head unit 1100 via a first connecting element 1110 and the inlet tube 710 may be connected to the motor housing 714 or the end unit 1140 via a second connecting element 1120. The connecting elements 1110 and 1120 may contain openings (or lumen) for receiving or delivering blood. The coupling may be done by gluing. In some embodiments, the inlet tube 710 and the first connecting element 1110 may be formed as a single piece. In some embodiments, the sensor head unit 1100 and the first connecting element 1110 may be formed as a single piece.

The sensor head unit 1100 of the MCS device 100 can include a tip including a number of sensors for measuring pressure and/or temperature.

The end unit 1140 may be a proximal end of the MCS device 100 and may form a transition between the motor housing 714 of the MCS device 100 and the shaft 306 for connecting the MCS device 100 to an external energy source or an external evaluation or control device (for example, the MCS controller 200 shown in FIG. 2).

The inlet tube 710 may include a guide cannula 1150, which at least partially has a structure or a surface structured at least partially along an extension direction. In some embodiments, the guide cannula 1150 can have a spiral-shaped surface structure. The guide cannula 1150 may include an electrical conducting element 1160 arranged inside the guide cannula 1150. The electrical conducting element 1160 may electrically connect the sensor head unit 1100 (and sensors located in or near the sensor head unit 1100) to the shaft 306 at the proximal end of the MCS device 100. In some embodiments, the electrical conducting element 1160 may contain a meander (for example, helically wound around the guide cannula 1150) in order to allow the inlet tube 710 to be bent at angle without causing damage to the electrical conducting element 1160. Additionally or alternatively, the meander may be placed in the area of the motor housing 714.

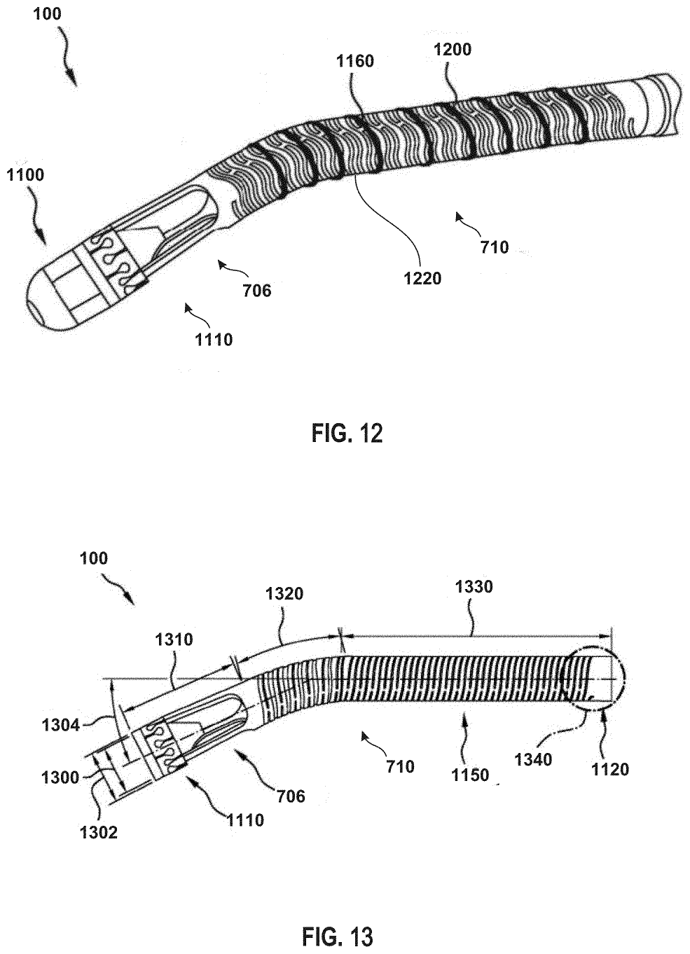

FIG. 12 shows another embodiment of a portion of the MCS device 100.

The MCS device 100 may include the sensor head unit 1100 and the inlet tube (or inlet cannula) 710. The sensor head unit 1100 may include a sensor assembly. From the head sensor unit 1100, the electrical conducting element 1160 may be guided along the inside of the inlet tube 710 from the first connection section 1110 through a pump inlet 706 and through a recess 1200 positioned proximate to the pump inlet 706 on a structural section 1220 abutting the outside of the inlet tube 710.

In some embodiments, the electrical conducting element 1160 may extend in a helical manner around the structural section 1220 along the longitudinal axis of the inlet tube 710. By extending as a continuous helix, the electrical conducting element 1160, which enables transmission of electrical data and energy the head sensor element 1100 and the pump of the MCS device 100 arranged downstream of the pump outlet (for example, outlet window/opening 708 shown in FIG. 7), can be fastened in a break-proof manner. For example, the electrical conducting element 1160 can be fastened by gluing, encapsulating, or casting

Additional details regarding the embodiments the MCS device and any related components and/or features shown and described with respect to FIG. 12 are described in PCT Publication No. WO2019229210, filed May 30, 2019, titled LINE DEVICE FOR CONDUCTING A BLOOD FLOW FOR A HEART SUPPORT SYSTEM, AND PRODUCTION AND ASSEMBLY METHOD, which is hereby incorporated by reference in its entirety.

FIG. 13 shows another embodiment of the inlet tube 710 of the MCS device 100. The inlet tube 710 may include the first connection section 1110 that may connect the inlet tube 710 to a distal tip (for example, sensor head unit 1100 shown in FIG. 11). The inlet tube 710 may additionally include the second connection section 1120 that may connect the inlet tube 710 to an impeller housing (for example, the impeller housing 922 shown in FIG. 9). The inlet tube 710 may additionally include the guide cannula (or structural section) 1150 extending between the second connection section 1120 and the first connection section 1110. In some embodiments, the guide cannula 1150 may extend between the inlet openings 706 and the first connection section 1110.

The guide cannula 1150 may include one or more stiffening recesses (for example, the recess 1200) that can change the rigidity of the inlet tube 710. The stiffening recesses may extend over a part of the guide cannula 1150 or over the entire guide cannula 1150. The stiffening recesses may be arranged in a helical circumferential manner. The stiffening recesses may be in the form of slots.

At the first connection section 1110, an inner diameter 1300 of the inlet tube 710 may be 6.5 millimeters (or between 4.5 to 8.5 millimeters) 1300. The outer diameter 1302 may be 7 millimeters (or between 5 mm to 9 mm). A bend angle between a proximal portion 1330 of the inlet tube 710 and a distal portion 1310 of the inlet tube may be 26 degrees (or between 16 degrees to 36 degrees). The distal portion 1310 may include the first connection section 1100 and the inlet openings 706, and a region of the guide cannula 1150 with a recess closest to the inlet openings 706. The length of the distal portion 1310 may be 15 millimeters (or between 10 millimeters and 20 millimeters). In some embodiments, the first connection section 1110 is part of the inlet openings 706. An adjacent bent portion 1320 of the guide cannula 1150 can be bent with respect to the longitudinal axis of the inlet tube 710 and may have a length of 14 millimeters. The proximal portion 1330 of the inlet tube 710 may include a remainder of the guide cannula 1150 and the second connection section 1120.

Additional details regarding the embodiment of the MCS device 100, the inlet tube 710, and any related components and/or features shown and described with respect to FIG. 13 are described in PCT Publication No. WO2019229210, filed May 30, 2019, titled LINE DEVICE FOR CONDUCTING A BLOOD FLOW FOR A HEART SUPPORT SYSTEM, AND PRODUCTION AND ASSEMBLY METHOD, which is hereby incorporated by reference in its entirety.

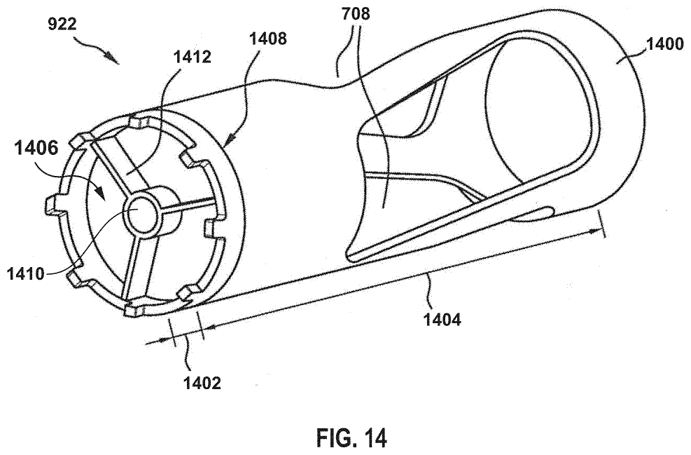

FIG. 14 shows another embodiment of the impeller housing 922. The impeller housing 922 may extend along an axis of rotation (for example, the axis 912 shown in FIG. 9A) in a longitudinal direction. The impeller housing 922 may include an impeller housing body 1400 extending in the longitudinal direction with a first longitudinal section 1402 and a second longitudinal section 1404 extending in the longitudinal direction. Furthermore, the impeller housing 922 may include at least one holder 1406, which may be arranged in the first longitudinal section 1402. The holder 1406 may include a bearing 1410 that can receive, for example, the second bearing 914 shown in FIG. 9A, for the rotatable mounting of the impeller 712 in the center of a transverse cross-section of the housing body 1400 through which, for example, blood flows. Additionally, the impeller housing 922 may include at least one outlet window/opening 708 arranged in the second longitudinal section 1404 and in a lateral surface of the impeller housing body 1400.

In some embodiments, the impeller housing body 1400 may be formed as a single piece. In some embodiments, the impeller housing body 1400 can be formed in several parts. A weld seam 1408 (running along the circumference of the body 1400) may be formed between the first longitudinal section 1402 and the second longitudinal section 1404 to attach them to one another.

In some embodiments, the holder 1406 and the first longitudinal section 1402 of the impeller housing body 1400 are formed as a single-piece (or integrated together). The holder 1406 may include, for example, three struts 1412 extending radially from the axis of rotation of the impeller (for example, the impeller 712) and supporting the bearing 1410 of the holder 1406.

The second longitudinal section 1404 of the impeller housing body 1400 may include a thin-walled tube in which the outlet openings 708 in the form of cutouts are provided. The outlet windows or openings 708 may be formed by laser cutting. The first longitudinal section 1402 of the impeller housing body 1400 can include a ring (not shown) with an inner and outer diameter of the thin-walled tube. In some embodiments, the ring may be a bearing star with at least one struts 1412.

In some embodiments, the holder 1406 may extend in the direction along the length the second longitudinal section 1404 beyond the first longitudinal section 1402. As a result, in some embodiments, the holder 1406 may align the first longitudinal section 1402 along the longitudinal axis of the impeller housing body 1400, which can facilitate the production of the weld seam 1408. In some embodiments, the struts 1412 may protrude proximally (downstream) or distally (upstream) over the ring and thereby describe the dimensions of the inner diameter of the tube. When joining the ring and the tube, the protruding connecting struts serve as a form-fitting centering. This can ensure that the bearing element (ring with integrated bearing star) is positioned concentrically with the pipe diameter.

The inlet tube 710 shown in FIG. 7 may be connected to the impeller housing 922, for example to the first longitudinal section 1402. In some embodiments, the inner diameters of the inlet tube 710, the first longitudinal section 1402, and the second longitudinal section 1404 may be equal, which may optimize or increase efficiency of the pump and minimize or reduce the likelihood of causing hemolysis. Optionally, the outer diameters of the inlet tube 710, the first longitudinal section 1402, and the second longitudinal section 1404 may be equal. The first longitudinal section 1402 may have a recess (not shown) that mates with an overlapping, corresponding recessed portion of the inlet tube 710 (not shown) wherein the sum of the thickness of the recesses of the first longitudinal section 1402 and the inlet tube 710 is substantially equal to the thickness of the non-recessed portions of the impeller housing 922 or the inlet tube 710. The struts 1410 of the holder 1406 may provide additional strength to a section of the MCS device 100 where the inlet tube 710 and the impeller housing 922 are joined. For example, the struts 1410 may maintain inner dimensions of the first longitudinal section 1402 and prevent or reduce the likelihood of the impeller housing 922 from deforming and contacting the impeller 712 housed within.

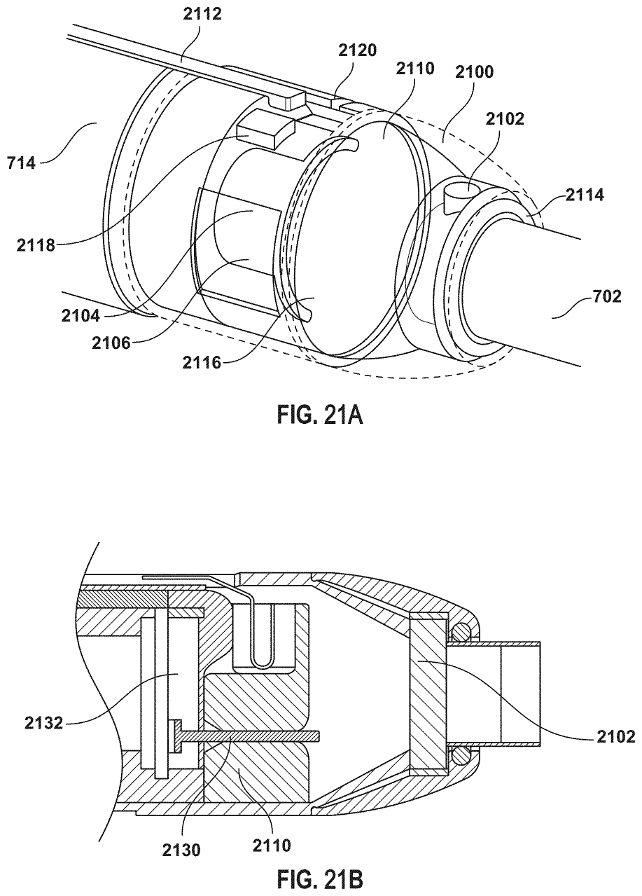

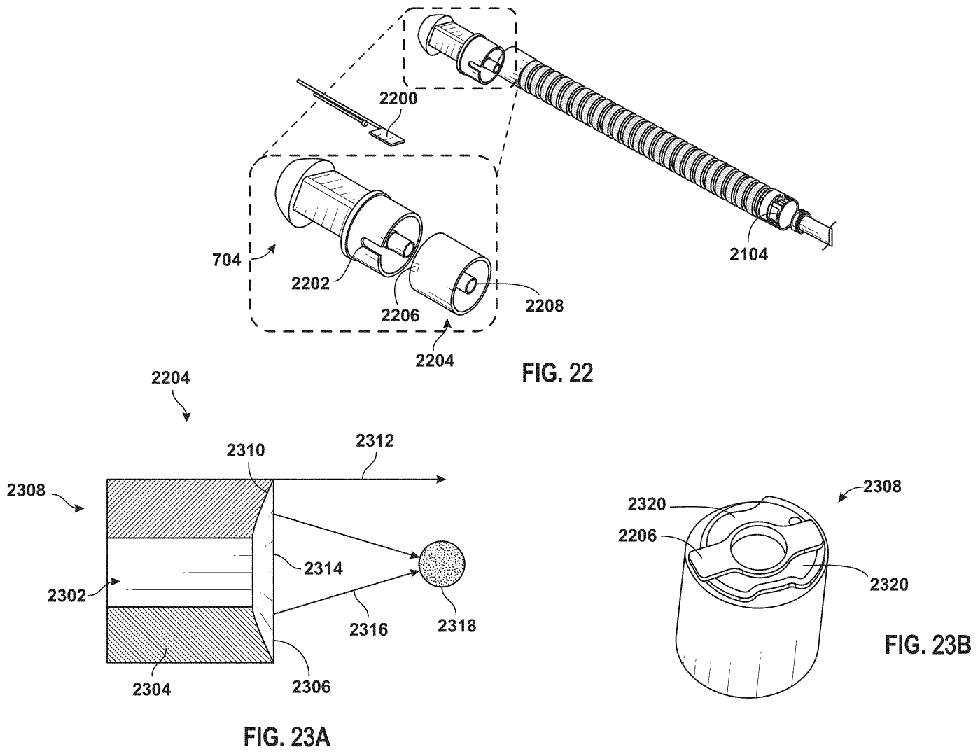

The struts 1410 of the holder 1406 may be used in a manufacturing process to align the inlet tube 710 (see FIG. 7) and associated features such as the ultrasound transducer 2204 shown in FIG. 22, laser cut slots, pathway for the electrical conducting element 1160 shown in FIG. 11, or inlet tube bend 1210 shown in FIG. 13 with the impeller housing 922. For example, the inlet tube 710 may have a mating notch or tab that mates with a mating notch or tab on the impeller housing 922, which may include the struts 1410 of the holder 1406, which may be radially unique so only one radial position allows the input tube 710 to couple with the impeller housing 922. The proximal end of the impeller housing 922 may have a mating feature such as a tab or notch that mates with a corresponding mating feature of the pump housing 940 shown in FIG. 9A (for example at outer diameter 962 shown in FIG. 9A) so that the impeller housing 922 may be radially aligned with the pump housing 940, which in turn is radially aligned with backend components for example illustrated in FIGS. 21A and 21B.

Additional details regarding the embodiment of the impeller housing 922 illustrated in FIG. 14 are described in PCT Publication No. WO2020011797, filed Jul. 9, 2019, titled IMPELLER HOUSING FOR AN IMPLANTABLE, VASCULAR SUPPORT SYSTEM, which is hereby incorporated by reference in its entirety.

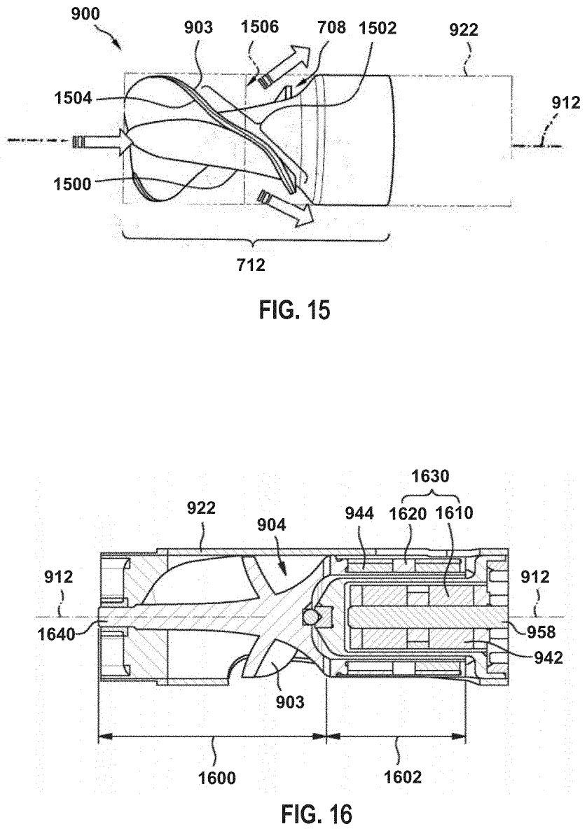

FIG. 15 shows a side, elevation view of another embodiment of the impeller 712. The impeller 712 may be rotatably mounted within an impeller housing 922 (not shown—see FIG. 9A). The impeller 712 may face outlet openings or windows 708. The impeller 712 provides for axial suction and radial or diagonal discharges of the blood via the outlet openings 708. The pump 900 can include an axis of rotation 912.

The impeller 712 may include at least one helically wound blade 903. The blade (or vane) 903 may ensure the efficient and gentle transport of blood (for example, flowing within the impeller housing 922 (not shown) and out via the outlet openings/windows 708. As shown in FIG. 15, the blade 903 may be helically wound around a hub 1500 of the pump 900. The hub 1500 may form an inner core of the impeller 712. In some embodiments, the hub 1500 may be the tapered portion 902 of the impeller 712 shown in FIG. 9A. A flow direction of the flow path through the pump 900 is indicated by three arrows. The blood is aspirated by a pump inlet (for example, the inlet openings 706 shown in FIG. 7) that acts as an intake opening upstream of the impeller 712.

In the embodiment of FIG. 15, a skeleton line 1504 of the blade 903 may include a point of inflection in a region of the upstream start of the outlet openings/windows 708.

In the embodiment illustrated in FIG. 15, the blade 903 may extend from an upstream end (for example, a distal end) of the impeller 712 over an entire length of the impeller 712 or at least over a portion or the majority of the hub 1500. The hub 1500 may have a diameter that increases in the direction of flow (indicated by the arrows), so that the diameter of the hub 1500 increases along the direction of flow. This shape of the hub 1500 may facilitate a radial and/or diagonal discharge of the blood. The blade 903 may include a blade section 1502 having a wave-shaped vane curvature, which is defined by a multiple curved portions of a skeleton line 1504 of the blade 903. As discussed herein, a wave-shaped curvature of the blade 903 can refer to a change in curvature of the blade section 1502 associated with at least one sign change (for example, from a positive change in curvature to a negative change in curvature, and vice versa).

In some embodiments, at least one section of the blade section 1502 is located opposite the outlet openings/windows 708.

In some embodiments, the blade section 1502 may at least partially be in the region of a flow-facing edge 1506 of the outlet opening 708. The blade section 1502 may represent a transition between a convex and a concave curvature of the skeleton line 1504 of the blades (or vanes) 903.

In some embodiments, the impeller 712 includes two blades 903 wound in the same direction around the hub 1500 and each having the blade section 1502. Alternatively, the impeller 712 may include more than two blades 903.

Additional details regarding the embodiment of the impeller illustrated in FIG. 14 are described in PCT Publication No. WO2019229223, filed May 30, 2019, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, which is hereby incorporated by reference in its entirety.

FIG. 16 shows another embodiment of the impeller 712 in the impeller housing 922 of another embodiment of the pump 900 with contactless torque transmission. The impeller 712 may include an impeller body 1640 rotatable about the axis of rotation 912 (see FIG. 9) with a first longitudinal section 1600 extending in the direction of the axis of rotation 912 and with a second longitudinal section 1602 extending in the direction of the axis of rotation 912. The impeller 712 may include at least one blade 903, which may be formed in the first longitudinal section 1600 and may convey a fluid (for example, blood) when rotated about the axis of rotation 912. In addition, the impeller 712 may include at least one magnet (for example, the second magnet array 944), which may be arranged in the second longitudinal section 1602. In the embodiment illustrated in FIG. 16, the impeller body 1640 may be formed in a single piece. In some embodiments, the first longitudinal section 1600 and the second longitudinal section 1602 of the impeller body 1640 are formed in one piece.

As shown in FIG. 16, the second longitudinal section 1602 may include a second rotor 1620 (for example, an outer rotor) for a magnetic coupling 1630 with a first rotor 1610 (for example, an inner rotor). The first rotor 1610 may include magnets (for example, the first magnet array 942 shown in FIG. 9A), which may be coupled to the drive shaft 958. The second rotor 1620 and the first rotor 1610 form the magnetic coupling 1630. In some embodiments, the magnetic coupling 1630 may be a radial coupling.

The amount of torque transmitted from the first rotor 1610 to the second rotor 1620 may depend on a number of factors. For example, the size of the magnets (for example, the first magnet array 942 and the second magnet array 944) may affect the amount of torque that can be transmitted from the first rotor 1610 to the second rotor 1620. In addition, larger magnets may be used to transmit greater amount of torque between the first rotor 1610 and the second rotor 1620. The size of the magnets may be limited by the space available in the pump 900. The distance between the magnets (for example, the first magnet array 942 and the second magnet array 944) and/or the distance between the motor shaft 958 and the impeller 712 can affect the amount of torque that can be transmitted from the first rotor 1610 to the second rotor 1620. For example, a smaller distance between the motor shaft 958 and the impeller 712 can result in greater amount of transmittable torque.

The amount of transmittable torque between the first rotor 1610 and the second rotor 1620 may also be affected by the arrangement and/or number of magnetic poles in the pump 900. The amount of transmittable torque can also be affected by material parameters such as energy density, remanence, coercive field strength, and/or saturation polarization.

Additional details regarding the embodiment of the pump 900 and the impeller 712 illustrated in FIG. 16 are described in PCT Publication No. WO2020011795, filed Jul. 9, 2019, titled IMPELLER FOR AN IMPLANTABLE, VASCULAR SUPPORT SYSTEM, which is hereby incorporated by reference in its entirety.

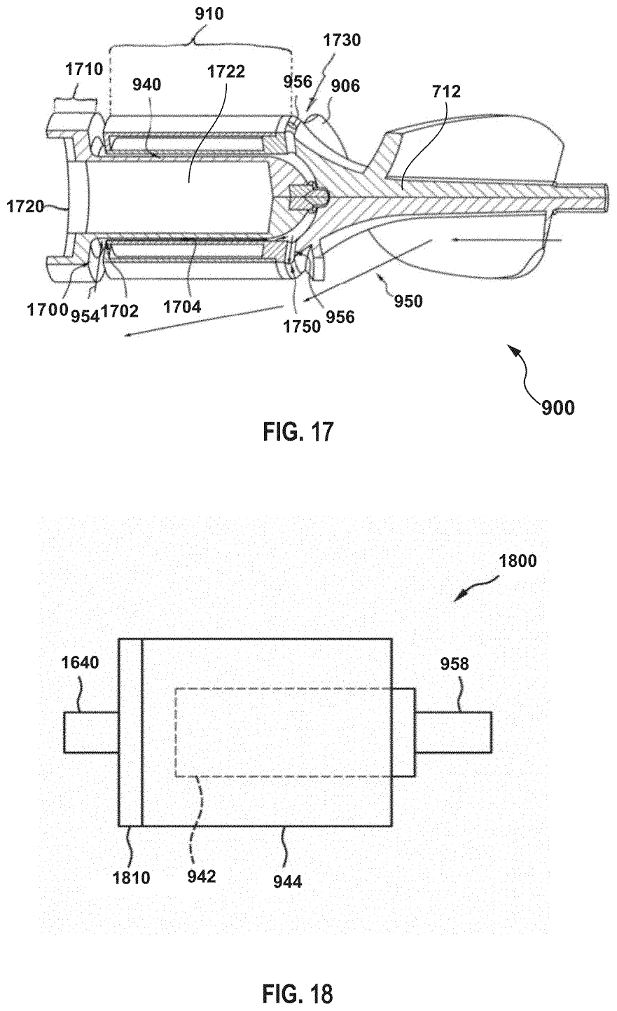

FIG. 17 shows another embodiment of the pump 900 with another embodiment of the impeller 712. A stand unit 1720 may be partially enclosed by the jacket 906 (see FIG. 9A) of the impeller 712. The stand unit 1720 may support the impeller 712 so that the impeller 712 can rotate about an axis of rotation of the stand unit 1720, which may be coaxial with a longitudinal axis of the impeller 712.

In a transition area or transition section 1730 between the blades (for example, the vanes 903 shown in FIG. 9A) of the impeller 712 and the jacket 906, one or more of the outlet openings/windows 708 may be arranged. The flow direction of a pump flow 950 and the flow path of a flushing or purging flow 954 (see FIG. 9A) are shown in FIG. 17.

The purging flow 954 may be introduced through a flushing inlet 1702 which may be a gap 1700 between a base 1710 of the standing unit 1720 and the jacket 906 of the impeller 712 surrounding a subsection 1722 of the standing unit 1720. The purging flow 954 may then be guided by, for example, centrifugal force generated by rotation of the impeller 712 through the intermediate space 1704 to one of the outlet openings of the flushing outlets 956 in order to flush the pump 900.

Additional details regarding the embodiment of the pump 900 and its components and/or features shown and described with respect to the FIG. 17 are described in PCT Publication No. WO2020030700, filed Aug. 7, 2019, titled BEARING DEVICE FOR A HEART SUPPORT SYSTEM, AND METHOD FOR RINSING A SPACE IN A BEARING DEVICE FOR A HEART SUPPORT SYSTEM, which is hereby incorporated by reference in its entirety.

FIG. 18 shows an alternative embodiment of a permanent magnetic radial rotary coupling 1800. In the illustrated embodiment, the coupling 1800 may include the first magnet array 942 and the second magnet array 944 shown in FIG. 9A (or the first rotor 1610 and the second rotor 1620 shown in FIG. 16).

In some embodiments, the first magnet array 942 (e.g., a drive magnet) and the second magnet array 944 (e.g., a driven magnet) are in the shape of a hollow cylinder. The driving shaft 958 can be arranged or positioned in the interior of the first magnet array 942.

The inner diameter of the second magnet array 944 can be larger than the outer diameter of the first magnet array 942. Optionally, the first magnet array 942 and the second magnet array 944 can be arranged coaxially. Both the first magnet array 942 and the second magnet array 944 can be mounted rotatably about a common axis. In some embodiments, the first magnet array 942 and the second magnet array 944 may include the same or different number of pole pairs. For example, both the first magnet array 942 and the second magnet array 944 each has two pole pairs. The first magnet array 942 may include four 90° segments in, for example, radial magnetization, while the second magnet array 944 can include eight 45° segments in a Halbach arrangement (or array). Alternatively, the first magnet array 942 can be magnetized in parallel and include one pole pair. Optionally, the first magnet array 942 can be diametrically magnetized. Likewise, the second magnet array 944 can include one pole pair. The second magnet array 944 may include segments in a Halbach arrangement, where the inner side of the second magnet array 944 may be the stronger side. As shown in FIG. 18, the first magnet array 942 is connected on one side to a driving shaft 958, while the second magnet array 944 on the other side is connected by means of an axial connecting ring 1810 to a driven shaft 1640. In some embodiments, the first magnet array 942 can be axially offset from the second magnet array 944 in order to generate an axial force.

In some embodiments, the driving shaft 958 can be connected to both axial ends of the first magnet array 942. The length (e.g., axial extension) of the first magnet array 942 can be smaller or greater than the length (e.g., axial extension) of the second magnet array 944. As such, both axial ends of the first magnet array 942 can be located inside or outside the second magnet array 944. In some embodiments, the first magnet array 942 may have following dimensions: an inner diameter of 1 mm, an outer diameter of 3 mm, and a magnetic thickness of 1 mm. In some embodiments, the second magnet array 944 may have the following dimensions: an inner diameter of 4 mm, an outer diameter of 5 mm, and a magnetic thickness of 0.5 mm.

Additional details regarding the embodiment magnetic coupling shown in FIG. 18 are described in PCT Publication No. WO2019219874, filed May 16, 2019, titled PERMANENT-MAGNETIC RADIAL ROTATING JOINT AND MICROPUMP COMPRISING SUCH A RADIAL ROTATING JOINT, which is hereby incorporated by reference in its entirety.

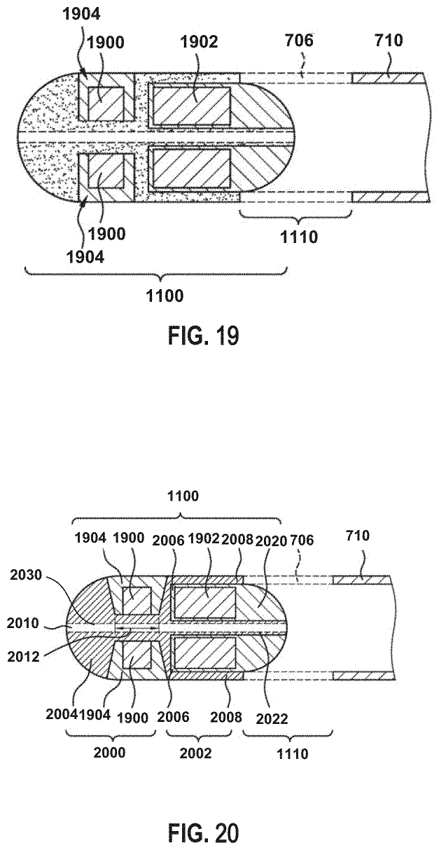

FIG. 19 shows an embodiment of the sensor head unit 1100 (see FIG. 11). The sensor head unit 1100 of the MCS device 100 may include a tip in the form of a sensor assembly which is used, for example, to measure the pressure and/or temperature of a patient. For this purpose, the sensor head unit 1100 may include one or more sensors 1900 and a signal transducer 1902.

In some embodiments, the two sensors 1900 may be a pressure sensor and/or a temperature sensor. The signal transducer 1902 maybe an ultrasonic element. In some embodiments, both sensors 1900 may be arranged in a sensor cavity 1904, which is filled with a potting compound to protect the sensors 1900 from blood and/or mechanical damage. For example, this potting compound may be a solid and/or gel-like silicone and/or a silicone oil. As shown in FIG. 19, the sensor head unit 1100 may be connected to the inlet tube 710 (see FIG. 7) via the connecting element 1110. The connecting element 1110 may include one or more inlet windows 706 through which blood enters the MCS device 100.

Additional details regarding the embodiments of the sensor head unit 1100 and related components and/or features shown and described with respect to the FIG. 19 are described in PCT Publication No. WO2019234146, filed Jun. 6, 2019, titled LINE DEVICE FOR A VENTRICULAR ASSIST DEVICE AND METHOD FOR PRODUCING A LINE DEVICE, which is hereby incorporated by reference in its entirety.

FIG. 20 shows another embodiment of the sensor head unit 1100. The sensor head unit 1100 may be arranged at or coupled to, for example, proximal end of the inlet tube 710. The sensor head unit 1100 may be electrically connected to other electronic devices (e.g., the MCS controller 200) by means of a connecting element (such as the conducting element 1160 shown in FIG. 11) and an electrically conductive element (not shown) of the inlet tube 710.

The sensor head unit 1100 may include a sensor carrier element 2004 which, for example, may form a base body of the sensor head unit 1100.

The sensor carrier element 2004 may be manufactured, for example, from a thermoplastic material, which can optionally contain a radiopaque material, using an injection molding process. Alternatively, the sensor carrier element 2004 may be manufactured using a machining manufacturing process.

The sensor carrier element 2004 may have one or more sensor cavities 1904 for accommodating the sensors 1900. The sensor cavities 1904 may extend circumferentially at least a portion of the sensor carrier element 2004 or the entire circumference of the sensor carrier element 2004. For example, the sensor cavity 1904 may extend about 330° circumferentially around the outer surface of the sensor head unit 1100.

The sensor carrier element 2004 may have a signal transducer cavity 2006 for receiving the signal transducer 1902. The signal transducer cavity 2006 and the signal transducer 1902 may be cylindrical in shape. The cavity 2206 may be dimensioned to receive the signal transducer 1902 such that the signal transducer 1902 may not be able to move once placed within the signal transducer cavity 2006.

The sensor 1900 may be a temperature and/or pressure sensor. In some embodiments, one of the sensors 1900 can be a temperature sensor and the other can be a pressure sensor. In some embodiments, the sensor 1900 can be a barometric absolute pressure sensor.

The signal transducer 1902 may include an ultrasonic element that can generate ultrasonic signals and a lens element 2020. The lens element 2020 may be placed on or cover the ultrasonic element. The lens element 2020 may be an ultrasonic lens. In some embodiments, the lens element 2020 may be made of silicone. The lens element 245 may cover at least a portion of the signal transducer cavity 2006.

In some embodiments, the signal transducer cavity 2006 is open in a different direction than the sensor cavity 1904. For example, in the illustrated embodiment, the signal transducer cavity 2006 is open in a direction facing the inlet tube 710 of the MCS device 100, so that the signal generated by the signal transducer 1902 may be pointed towards the flow path of the inlet tube 710.

In some embodiments, the sensor cavity 1904 is wider towards the outside (near the outer circumference of the sensor head unit 1100) than at its base (towards the center of the sensor head unit 1100). This configuration of the sensor cavity 1904 may be advantageous during the manufacturing process of the sensor head unit 1100. The sensor 1900 may be placed inside the sensor cavity 1904 and silicone may be introduced into the cavity as a seal. As silicone cures and swells, the wider portion of the sensor cavity 1904 reduces tensioning force applied to sensor elements of the sensors 1900 (for example, a MEMS element).

The outer end 2000 of the sensor head unit 1100 may be round in order to prevent or reduce likelihood of injuries during installation of the MCS device 100. Moreover, the rounded tip of the outer end 2000 may allow the MCS device to slide well during installation.

In some embodiments, the sensor carrier element 2004 may include a channel 2010. The channel 2010 may receive a guidewire (not shown). When the MCS device 100 is implanted, for example, into the left ventricle or the aorta of a patient, the guidewire is first placed into the patient's ventricle. The MCS device 100 is then pushed onto the guidewire and advanced along the guidewire to the end position.

In some embodiments, the sensor carrier 2004 can contain a tubular continuation 2022 which may extend far enough into the signal transducer cavity so that a guidewire may be guided to a contact surface between the ultrasonic lens 2020 and the blood in the connecting element 1100 (see FIG. 11).

In some embodiments, a tube 2030 (for example metallic, for example made of stainless steel, titanium or nitinol) may be inserted into the channel 2010, which extends through the sensor carrier element 2004 and beyond into the signal transducer cavity 2006 up to the contact surface between the ultrasonic lens 2020 and blood in the area of the connecting element 1100 (see FIG. 11). This can allow for a significantly smaller wall thickness, which may result in a reduced central opening width around the signal transducer 1902, which is particularly advantageous when the signal transducer 1902 is an ultrasonic transducer. The signal transducer cavity 2006 may be surrounded by a jacket 2008, which may be a part of the sensor carrier element 2004. The signal transducer 1902 may be pushed into the signal transducer cavity 2006. The gaps between the tubular continuation 2022 and the signal transducer 1902, or the gaps between the tube 2030 and the signal transducer 1902, as well as the gap between the signal transducer 1902 and the jacket 2008 may be filled with epoxy or silicone, for example, which also may serve as an adhesive.

The outer end 2000 may provide additional protection for the sensors 1900 arranged in the sensor cavity 1904.

In some embodiments, the sensor carrier element 2004 may include a web 2012 positioned between the sensors 1900. In order to place the sensors 1900 around the web 2012, the sensors 1900 may be arranged on a flexible printed circuit board (PCB) or a thin-film substrate, which then can be placed on the web 2012. For example, the sensors 1900 may be bonded to a thin-film substrate. In some embodiments, the thin-film substrate may be supported with stiffening element so that it would not bend in the area with the sensors 1900. The thin-film substrate may or may not be cylindrical. In some embodiments, the thin-film substrate may be a cuboid with rounded corners. The rounded corners of the web 2012 serve, for example, to maintain the bending radii of the thin-film substrate (for example, a polyimide-gold layer structure).

In some embodiments, the sensor cavity 1904 may be filled with a potting compound or a casting compound to protect the sensors 1900 from blood and mechanical damage. The casting compound may be a solid and/or gel-like silicone or a silicone oil. The potting compound or the casting compound may allow accurate pressure measurements by the sensors 1900.

Additional details regarding the embodiments of the sensor head unit 1100 and related components and/or features shown and described with respect to the of FIG. 20 are described in PCT Publication No. WO2019234149, filed Jun. 6, 2019, titled SENSOR HEAD DEVICE FOR A MINIMAL INVASIVE VENTRICULAR ASSIST DEVICE AND METHOD FOR PRODUCING SUCH A SENSOR HEAD DEVICE, which is hereby incorporated by reference in its entirety.

Referring to FIGS. 21A and 21B, a junction between the proximal end of the MCS device 100 (for example, a proximal end of the motor housing 714 shown in FIG. 7) and the catheter shaft 306 (see FIG. 7). The junction may include a backend adaptor 2120 enclosing electrical components and connections in the junction. The backend adaptor 2120 may be connected to the motor housing 714. A strain relief 702 (also referred to as a bend relief) may be a laser cut metallic tube (e.g., Nitinol) and contains a distal section of the shaft 306 and may be connected to the backend adaptor 2120 with a backend pin 2102 inserted through a hole in the strain relief 702 and the backend adaptor 2120. The strain relief 702 may provide sufficient tensile strength to pull on the shaft 306 to remove or manipulate the MCS device 100. A backend sealing cover 2100 may cover a portion of the backend adaptor 2120, particularly where the backend adaptor 2120 connects to the strain relief 702. A seal 2114 (such as an O-ring gasket) may be provided between the sealing cover 2100 and the strain relief 702, to form a seal to enclose and separate various electrical connections and components on the proximal end of the pump 900 from external environments.

In some embodiments, the pump 900 may be secured to the catheter shaft 306 via other suitable methods that provide sufficient strength under tension to resist detachment. In the embodiment illustrated in FIGS. 21A and 21B, a mechanical engagement between the proximal end of the MCS device 100 (or proximal end of the motor housing 714) and the shaft 306 is provided by a transversely extending back-end pin 2102 interlocking with the catheter shaft 306 and the proximal end of the pump 900.

The backend adapter 2120 of the MCS device 100 may include a sensor window 2106, which can allow pressure and/or temperature signals to propagate through and into an internal MEMS sensor 2104. The sensor window 2106 may be sealed closed with a pressure and temperature conducting material such as a thin layer of silicone. A cable and data interface 2110 (which may be a molded interconnect device) may be provided, to facilitate assembly and provide electrical communication between conductors in the catheter shaft 306 and electronics in the pump 900. Soldering terminals 2118 may be provided for soldering the conductors of the catheter shaft 306 and may be in electrical communication via electrical traces within the cable and data interface 2110 to the motor or sensors of the MCS device 100. A flexible PCB connector 2116 may communicate with supply and discharge conductors carried on a flexible PCB support 2112 to the distal end of the MCS device 100 to connect to sensors (e.g., MEMS pressure and temperature sensors, ultrasound transducer) and to proximal sensors 2104. As shown in FIG. 21A, the flexible PCB support 2112 may be positioned at least partially along the motor housing 714. The flexible PCB support 2112 may extend distally along at least a portion of the length of the MCS device 100 or the motor housing 714, to the distal sensors or transducers of the MCS device 100. The flexible PCB support 2112 may extend in a helical fashion around the inlet tube 710 (see FIG. 7) to preserve flexibility of the inlet tube 710. The cable and data interface 2110 can also provide for electrical communication between conductors in the catheter shaft 306 and a motor PCB 2132. One or more motor pins 2130 can be coupled to (for example, inserted into) the cable and data interface 2110.

Referring to FIG. 22, the distal end of the MCS device 100 may be provided with at least one sensor such as a distal MEMS sensor 2200 for monitoring pressure and or temperature in, for example, the left ventricle. The sensor 2200 may be positioned on sidewall of a distal end of the MCS device 100 with a sensor surface oriented substantially parallel to a longitudinal axis of the MCS device 100. In some embodiments, the sensor 2200 may be positioned within sidewall of the MCS device 100 and laterally facing a window in the sidewall such as illustrated in FIG. 22. The MEMS sensor 2200 may be positioned on the nose piece or distal tip 704 (see FIG. 7), distally of an annular support flange and distally of the blood intake ports (for example, inlet windows 706). As described herein, another MEMS sensor (for example, the proximal MEMS sensor 2104 shown in FIG. 10A) can be positioned within or near the proximal end of the MCS device 100.

An ultrasound transducer (or an ultrasonic sensor) 2204 may be provided distally of the blood intake port (for example, inlet windows 706). The ultrasound transducer 2204 may include a positioning tab 2206 configured to couple with a positioning channel 2202 of the nose piece 704. The positioning channel 2202 may be positioned proximally of the sensor 2200. A guidewire lumen 2208 can extend through the transducer 2204. Further details regarding the transducer 2204 are discussed in connection with FIGS. 23A and 23B. The ultrasound transducer 2204 may include an acoustical backing 2304, a proximal concave surface 2306, and a distal end surface 2308. The guidewire lumen 2302 may extend through the acoustical backing 2304. The proximal concave surface 2306 may be provided with at least one and preferably two or more piezo elements 2310, focused for convergence at a focal distance 2312 within the range of from about 6 mm to about 14 mm from the concave surface 2306. In some embodiments, the focal distance 2312 of the piezo elements 2310 may be about 10 mm. It is contemplated that the focal distance 2312 may be greater than or less than 10 mm. The piezo elements 2310 on the concave surface 2306 may direct ultrasonic waves 2316 to a focus region 2318 positioned at the focal distance 2312. In some embodiments, the concave surface 2306 and piezo elements 2310 may be covered by an acoustical impedance matching layer 2314.

The distal end 2308 of the transducer 2204 may be provided with a plurality of electrodes 2320, to connect conductors to the piezo elements 2310. In addition, a positioning structure such as a tab or recess, such as for example, the positioning tab 2206 shown in FIG. 22, may be provided to ensure appropriate rotational orientation of the ultrasound transducer 2204 by engaging a complementary tab or recess, such as the positioning channel 2202 shown in FIG. 22, in the adjacent structure such as the nose piece 704 or a housing for the MCS device 100. In some embodiments, the focus region 2318 of the directed ultrasound waves 2316 may therefore be positioned in the blood flow path adjacent to or downstream of the blood intake ports (for example, inlet windows 706 shown in FIG. 7) within the blood flow channel of the pump 900. This may allow the transducer 2204 to provide blood flow velocity data by assessing Doppler shift of the reflected ultrasound waves detected at the focus region 2318.

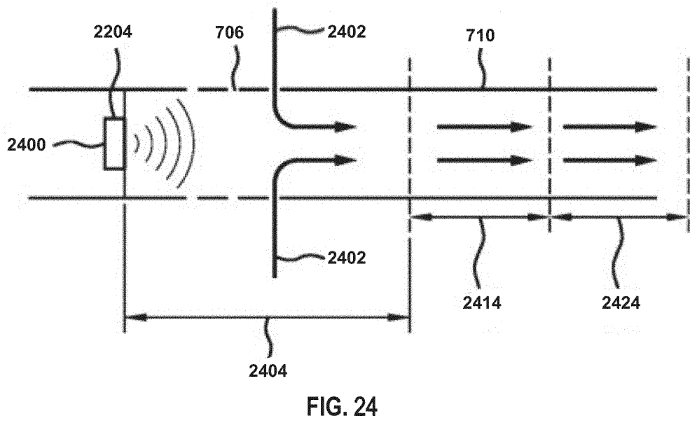

FIG. 24 shows an example schematic illustration of a Doppler measurement of a fluid flowing through the MCS device 100. As shown in FIG. 24, an ultrasonic transducer 2204 (see FIG. 22) can be used to carry out a Doppler measurement in an inlet tube 710 of the MCS device 100. During operation, a fluid volume flow 2402 enters the MCS device 100 through one or more of the inlet openings 706.

A measurement window, also referred to as an observation window and/or a measurement area, may be an area for taking ultrasound measurements (for example, Doppler measurements). A first measurement window 2414 and a second measurement window 2424 are positioned along a lumen of the inlet tube 710. The location of a measurement window (for example, the first measurement window 2414 or the second measurement window 2424) may depend on the specific configurations of the MCS device 100 and may be placed where suitable flow conditions exist. As shown in FIG. 24, there are no parallel flow lines in the area 2404 located to the left of the measurement window 2414. Since the Doppler effect depends on cos(a) between the main beam direction of the ultrasonic transducer 2204 and a direction of flow inside the inlet tube 710, it is advantageous to measure in an area where fluid flow lines are parallel to beam direction of the ultrasonic transducer 2204. As such, the first measurement window 2414 may be a better location than the area 2404 to take Doppler measurements. Taking Doppler measurements in a measurement window that is too far away (for example, the second measurement window 2424) is possible in principle, but can exacerbate an aliasing effect and/or provide strong attenuation of the ultrasonic signal, thereby resulting in less accurate measurements.

The ultrasonic transducer 2204 may include one or more ultrasound (transducer) elements 2400 (that is, elements that generate ultrasound beams). In some embodiments, the ultrasonic transducer 2204 may perform pulsed Doppler measurements. The Doppler measurements may be taken randomly or at regular intervals.

The suitable measurement window (for example, the first measurement window 2414 in the example illustrated in FIG. 24) may be determined by a Pulsed Wave Doppler (PWD) system prior to taking measurements and measurements taken at various depths (that is, distances from the ultrasound transducer 2204) may be used to determine flow conditions in the inlet tube 710.

Additional details regarding the embodiments of the Doppler measurement scheme and any related components and/or features shown and described with respect to FIG. 24 are described in PCT Publication No. WO2019234166, filed Jun. 6, 2019, titled METHOD FOR DETERMINING A FLOW SPEED OF A FLUID FLOWING THROUGH AN IMPLANTED, VASCULAR ASSISTANCE SYSTEM AND IMPLANTABLE, VASCULAR ASSISTANCE SYSTEM, which is hereby incorporated by reference in its entirety.

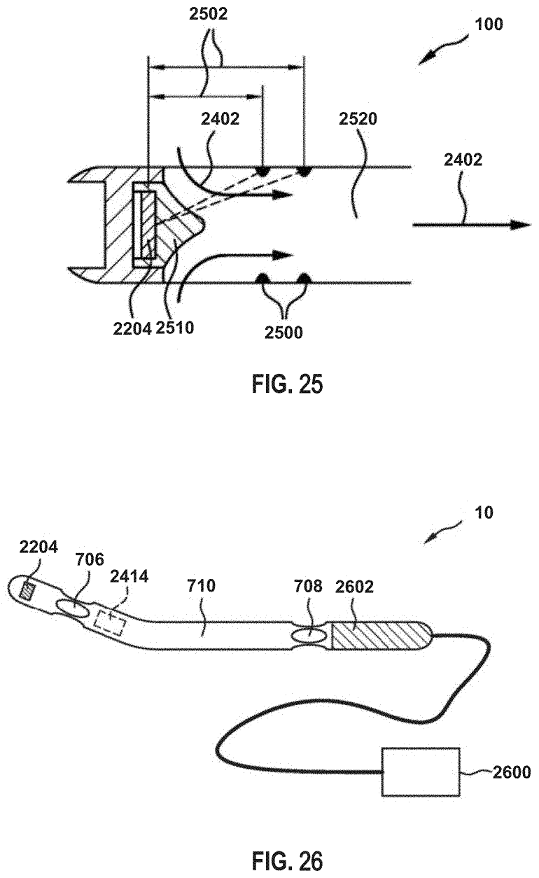

FIG. 25 shows a cross-sectional, schematic view of a distal region of another embodiment of the MCS device 100. The fluid volume flow 2402 may present an example fluid flow direction through, for example, the inlet openings 706 of the MCS device 100. The MCS device 100 may include the ultrasonic transducer 2204 as described herein. The MCS device 100 may include two sets of sound reflectors 2500 positioned around an inner circumference of a flow channel (for example, the inlet tube 710) of the MCS device 100. In some embodiments, the sound reflector 2500 may be arranged in the field of view 2520 of the ultrasonic transducer 2204. In some embodiments, the sound reflectors 2500 may each be positioned at a corresponding defined distances 2502 from the ultrasonic transducer 2204. As shown in FIG. 25, the flow channel may be formed inside the inlet tube 710 of the MCS device 100 (see FIG. 7).

In some embodiments, the MCS device 100 may include a flow guide body 2510 that may be placed, for example, directly in front of the ultrasonic transducer 2204. The flow guide 2510 may not be spaced apart from the ultrasonic transducer 2204 and may be permeable to ultrasonic signals. The fluid volume flow 2402 flows in the direction of the pump. The tip of the MCS device (for example, the distal pump region 700 shown in FIG. 7) shown in FIG. 25 can protrude in a preferred arrangement with the end shown here on the left into a ventricle (not shown here) of a heart, with the pump at least partially in the aorta (not shown here). With this arrangement, the MCS device thus penetrates an aortic valve (not shown here).

The ultrasonic transducer 2204, in particular an ultrasonic element of the ultrasonic transducer 2204, is usually placed such that the angle between the line of sight associated with the ultrasonic transducer 2204 and the flow direction inside the flow channel as described herein is approximately zero degrees.

Optionally, the flow guide 2510 may be overlayed on a lens associated with the ultrasonic transducer 2204 (for example, the lens element 2020 (see FIG. 20). If the ultrasonic transduce 2204 includes a concave surface (for example, the concave surface 2306), the flow guide 2510 may be placed on the concave surface.

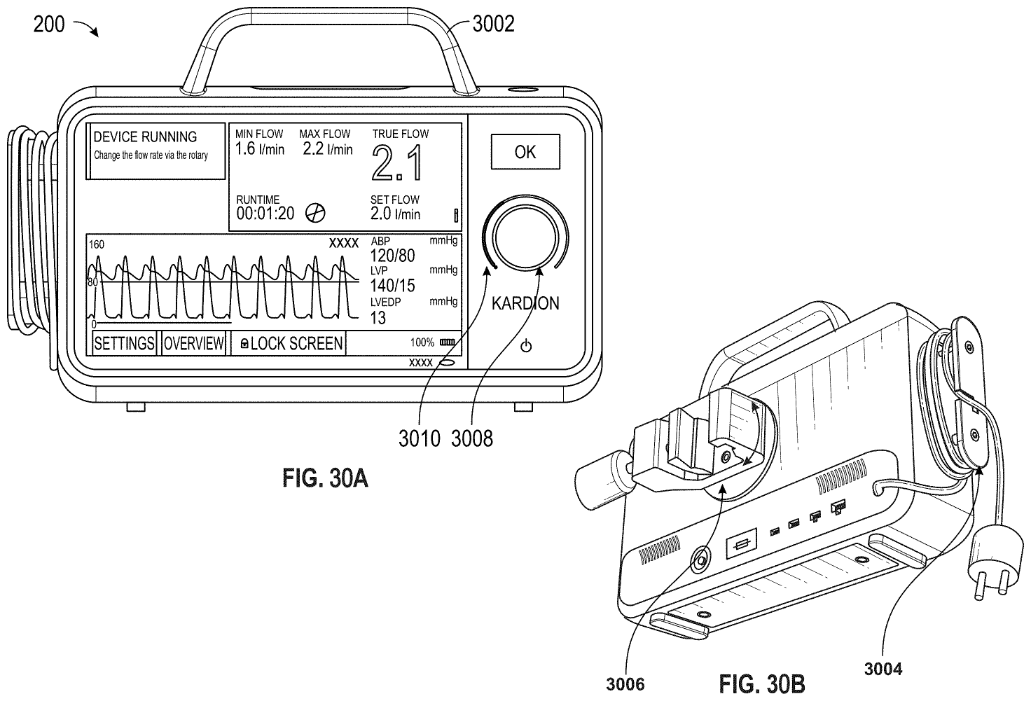

Additional details regarding the embodiments of the MCS device 100 and any related components and/or features (for example, related to Doppler measurements) shown or described with respect to FIG. 30 are described in PCT Publication No. WO2019234163, filed Jun. 6, 2019, titled METHOD AND SYSTEM FOR DETERMINING THE SPEED OF SOUND IN A FLUID IN THE REGION OF AN IMPLANTED VASCULAR SUPPORT SYSTEM, which is hereby incorporated by reference in its entirety.

FIG. 26 shows another embodiment of the MCS system 10 with the MCS device 100. The MCS system 10 may include the ultrasonic transducer 2204 that can carry out pulsed Doppler measurements with different pulse repetition rates. The MCS system 10 may include a processing unit 2600 (for example, the MCS controller 200 shown in FIG. 2) that can determine a flow speed of a fluid (e.g., blood) flowing through the MCS device 100 using measurement results of the pulsed Doppler measurements. In some embodiments, as described herein, the ultrasonic transducer 2204 may be integrated in a distal tip of the inlet tube 710 of the MCS device 100.

The ultrasonic transducer 2204 may determine the flow velocity (amount and at least one direction) of a fluid (for example, blood) flowing through the MCS device 100 and/or the fluid volume flow, which may be referred to as pump volume flow (Qp). In some embodiments, the ultrasonic transducer 2204 may perform pulsed Doppler measurements of the fluid flowing within the inlet tube 710. As described herein, the fluid (for example, blood) can enter the interior of the inlet tube 710 through one or more of the inlet openings 706 (for example, from the ventricle) and exit through one or more of the outlet windows or openings 708 (for example, into the aorta). The flow through the inlet tube 710 may be generated by a motor 2602.

Additional details regarding the embodiments of the MCS system 10, the MCS device 100, and any related components and/or features shown and described with respect to FIG. 26 are described in PCT Publication No. WO2019234164, filed Jun. 6, 2019, titled METHOD FOR DETERMINING A FLOW RATE OF A FLUID FLOWING THROUGH AN IMPLANTED VASCULAR SUPPORT SYSTEM, AND IMPLANTABLE VASCULAR SUPPORT SYSTEM, which is hereby incorporated by reference in its entirety.

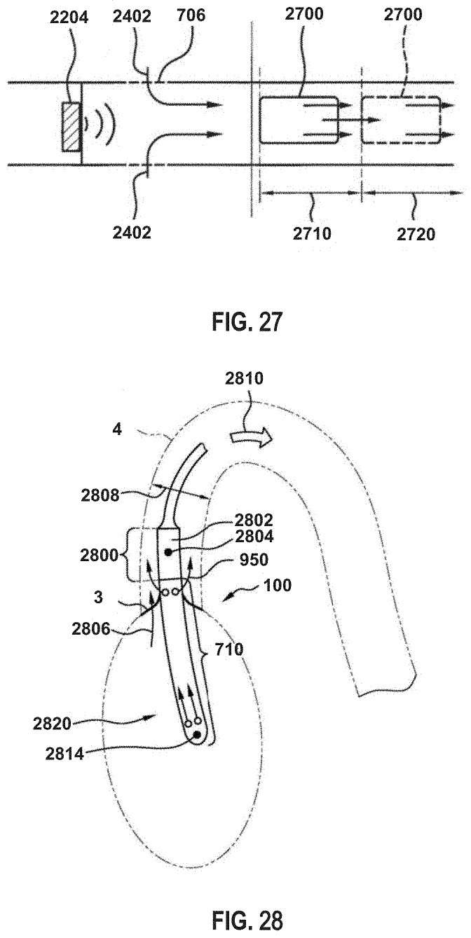

FIG. 27 shows an example schematic illustration of an example method of determining the amount fluid flowing though the MCS device 100 using Doppler measurements. The MCS device 100 may include an ultrasonic transducer (or an ultrasonic sensor) 2204 that can emit, for example, ultrasonic beams in the direction of fluid flow. In the area approximate to the inlet openings 706 of the MCS device 100, the fluid flow 2402 (for example, blood flow) does not yet show a constant flow profile. Downstream, in the areas 2710, 2720 (similar to the first and the second measurement windows 2414, 2424 shown in FIG. 24), however, the radial flow profile may be largely constant. Thus, an observation window 2700 of the ultrasonic transducer 2204 can advantageously be shifted with an observation window speed VGate. In some embodiments, the areas 2710, 2720 can lie in a channel (or a lumen) of an inlet tube (for example, the inlet tube 710 shown in FIG. 7).

If, for example, as shown in the following equation (1), with a pulse repetition frequency (PRF) of 25 kHz and an ultrasonic frequency of fo=4 MHz, a flow velocity of V_{Blut}=3 m/s away from the piezo element of the ultrasonic transducer 2204 in a fixed observation window is to be measured, this leads to a Doppler shift of −15.58 kHz. With the given PRF of 25 kHz and the evaluation of positive and negative velocities, this Doppler shift can no longer be represented in the negative part of the Doppler spectrum and is accordingly represented as 9.42 kHz in the positive frequency range of the spectrum.

If, however, the observation window 2700 is moved away from the piezo element of the ultrasonic transducer 2204 at a displacement speed of, for example, VGate=1.75 m/s, the resulting (or relative) flow speed is reduced, here as an example reduced to 3 m/s−1.75 m/s=1.25 m/s.

The resulting Doppler shift of −6.49 kHz can be represented without ambiguity in the Doppler spectrum at a PRF of 25 kHz (see equation (4) below).

$$\begin{matrix}{f}_{d , wrapped} = \frac{- 2 * {v}_{Blut} * {f}_{0}}{{c}_{0}} & ( 1 ) \\ = \frac{- 2 * 3 \frac{m}{s} * 4 Mhz}{1540 \frac{m}{s}} & ( 2 ) \\ = - 15.58 kHz & ( 3 ) \\ {f}_{d , trackin {g}_{d} oppl} = \frac{- 2 * ( {v}_{Blut} - {v}_{Gate} ) * {f}_{0}}{{c}_{0}} & ( 4 ) \\ = \frac{- 2 * ( 3 \frac{m}{s} - 1.75 \frac{m}{s} ) * 4 MHz}{1540 \frac{m}{s}} & ( 5 ) \\ = - 6.49 kHz & ( 6 )\end{matrix}$$

This represents an example of how the observation window speed can be determined in such a way that a Doppler shift is transformed into a range that can be represented without ambiguity. A determination of the observation window speed can be made using previous estimates of the flow speed of the blood through the MCS device 100. In some embodiments, such estimates can be made based on previously performed ultrasound measurements (e.g., using a fixed observation window) using the ultrasonic transducer 2204 of the MCS device 100. In some embodiments, estimates may be made based on an empirical value based on the patient's age, the severity of the patient's illness, and other factors.

Additional details regarding the embodiments of FIG. 27 are described in PCT Publication No. WO2020064707, filed Sep. 24, 2019, titled METHOD AND SYSTEM FOR DETERMINING A FLOW SPEED OF A FLUID FLOWING THROUGH AN IMPLANTED, VASCULAR ASSISTANCE SYSTEM, which is hereby incorporated by reference in its entirety.

FIG. 28 shows another embodiment of the MCS device 100 with temperatures sensors to determine blood flow velocity. The MCS device 100 may include a hose-like elongated structure with a cannula section in which the inlet tube 710 is designed as a cannula. The MCS device 100 may include a motor housing section 2800 (for example, the motor housing 714 shown in FIG. 7) connected to the inlet tube 710, and a motor 2802 may be located in the motor housing 2800.

The MCS device 100 may protrude from the aorta 4 through the aortic valve 3 distally into the ventricle 2 (see FIG. 1). The MCS device 100 may include, for example, an inlet tube 710, which may protrude into the ventricle 2. A fluid volume flow 950 (see FIG. 9) is conveyed, for example pumped, from the ventricle 2 into the aorta 4 through the inlet tube 710 using the motor 2800 of the MCS device 100. The fluid volume flow 950 is therefore also referred to as the pump volume flow (QP′), which quantifies only the flow through the MCS device 100.

However, as shown in FIG. 28, a certain aortic valve volume flow 2806 reaches the aorta 4 via the physiological path through the aortic valve 3. The cardiac output or the total fluid volume flow 2810 (Q_{H}ZV) passing through a cross-sectional geometry 2808 of the aorta 4 in the area of the MCS device 100 from the ventricle 2 to the aorta 4 is accordingly the sum of the fluid volume flow 950 (QP′) and aortic valve volume flow 2806 (Qa), which is described by equation (7) below.

$${Q}_{H} Z V = {Q}_{P} + {Q}_{a} \qquad ( 7 )$$

The MCS device 100 may include a reference temperature sensor 2814 for determining a reference temperature of blood. Additionally, the MCS device 100 may include the motor 2802 and a motor temperature sensor 2804 for determining a motor temperature of the electric motor 2802. Optionally, the MCS device 100 may include a current sensor (not shown) for determining the thermal power loss (not shown) of the electric motor 2802.

The motor temperature sensor 2804 may be integrated in a motor housing 2800 in which the thermal power loss of the electric motor 2802 may be dissipated to the surrounding fluid. The motor temperature sensor 2804 may be set up and arranged in such a way that it can measure the motor temperature. In some embodiments, the motor temperature sensor 2804 may be set up and arranged such that it measures a surface temperature of the motor housing 2800 or a temperature of the stator (not shown) of the electric motor 2802. Here, the temperature of the stator can be approximated from an internal temperature in the motor housing 2800 between the motor housing 2800 and the winding package (not shown). Optionally, the temperature in the winding package can also be measured directly using a separate temperature sensor.

The reference temperature sensor 2814 may detect the reference temperature, which may be the background blood temperature as an example. For example, the reference temperature sensor 2814 may be placed in a thermally unaffected blood flow in front (for example, upstream) of the electric motor 2802, where the electric motor 2802 represents the heat source. Optionally, the reference temperature sensor 2814 may be arranged in a region of the inlet tube (for example, the inlet tube 710) at a distance from the motor housing 2800. For example, the reference temperature sensor 2814 may be positioned at a distal end of the inlet tube 710 (for example, where the blood flows from the ventricle 2 into the inlet tube 710).

Additional details regarding the embodiments the MCS device 100 and any related devices and/or features described or shown with respect to FIG. 28 are described in PCT Publication No. WO2019234162, filed Jun. 6, 2019, titled METHOD FOR DETERMINING A FLUID TOTAL VOLUME FLOW IN THE REGION OF AN IMPLANTED VASCULAR SUPPORT SYSTEM AND IMPLANTABLE VASCULAR SUPPORT SYSTEM, which is hereby incorporated by reference in its entirety.

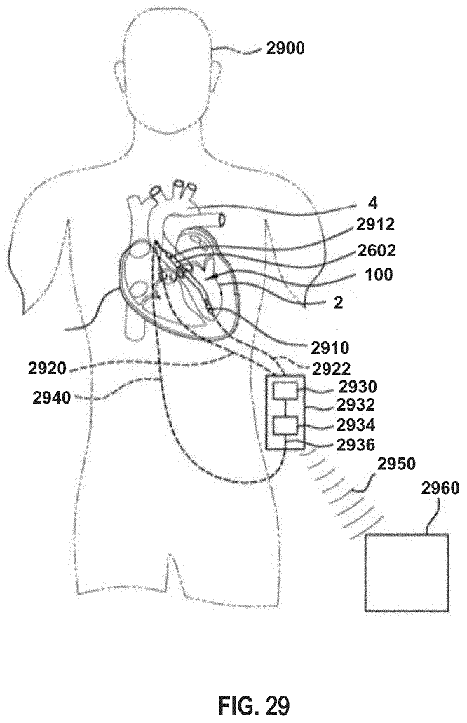

FIG. 29 shows another embodiment of the MCS system 10 implanted in a patient 2900. The MCS system 10 may interact with a monitoring device 2932 for monitoring a state of health of the patient 2900. The MCS system 10 may include the MCS device 100 described herein, which can pump blood from the ventricle 2 of the heart 1 into the aorta 4 of the patient 2900. The MCS device 100 may include a first pressure sensor 2910 and a second pressure sensor 2912. The first pressure sensor 2910 may generate and send a first pressure signal 2920 to the monitoring device 2932. The second pressure sensor 2912 can send a second pressure signal 2922 to the monitoring device 2932. The pressure signals 2920, 2922 may be transmitted to the monitoring device 2932 wirelessly or via a wire or a cable.

The first pressure sensor 2910 and the second pressure sensor 2912 may be positioned at a predetermined distance from one another in the MCS device 100, so that they can detect, for example, the blood pressure, blood pressure fluctuations or a pulse wave of blood. The monitoring device 2932 may include a reading interface (or input/output interface) 2930 which may receive the first pressure signal 2920 and the second pressure signal 2922. The pressure signals 2920, 2922, once received, may be forwarded to a processing unit 2934, which may then determine a processing value 2936. The processing value 2936 may then be used to determine a state of health of the patient 2900. By monitoring and tracking the processing values 2936, the state of health of the patient 2900 may be monitored.

In some embodiments, the processing value 2936 may be a transit time of the pulse wave of blood between the first pressure sensor 2910 and the second pressure sensor 2912. Alternatively or additionally, such a processing value 2936 may also represent a parameter that represents an elasticity of vessel walls such as a wall of the aorta 4. As such the processing value 2936, for example, may be used to determine the patient's state of health with regard to the elasticity of vessel walls. For example, the processing value 2936 may be used to determine or estimate the amount of deposits or calcifications on the inner walls of the vessels.

The monitoring device 2932 may generate a control signal 2940 based on the processing value 2936. For example, the control signal 2940 may control the MS device 100 of the MCS system 10 in order to provide sufficient amount of blood flow or generate an artificial increase in blood pressure to allow the patient 2900 to participate in a desired or specific activity (for example, climbing stairs).

The monitoring device 2932 may generate and transmit (wirelessly or via a wire) a signal 2950 to a separate computer unit 2960 (for example, a data server such as a cloud server) based on the processing value 2936, the pressure signals 2920, 2922, and/or the control signal 2940. The signal 2950 may include a notification or an evaluation related to the pressure signals 2920, 2922, the processing value 2936, and/or the control signal 2940. In some embodiments, the monitoring device 2932 may be worn externally to the patient 2900. For example, the monitoring device 2932 may be attached to a belt of the patient 2900. Alternatively, the monitoring device 2932 may be an integral component of the MCS system 10, such that the pressure signals 2920, 2922 may be transmitted to the monitoring device 2932 via wires or cables. If the monitoring device is implanted in the patient 2900, the monitoring device 2932 may include an energy storage device (for example, a long-life, rechargeable battery). The energy storage device (not shown) of the monitoring device 2932 may be charged via a power, supplying cable or a wireless power transmission system.

In some embodiments, the monitoring device 2932 may be divided into multiple components. For example, the reading interface 2930 may be implanted in the patient 2900 while the processing unit 2934 may be positioned externally (for example, worn on a belt of the patient 2900), where the reading interface 2930 and the processing unit 2934 may wirelessly communicate with one another.

In some embodiments, one of the pressure sensors 2910, 2912 may be arranged outside the patient 2900. For example, one of the pressure sensors 2910, 2912 may be placed in the monitoring device 2932. The pressure value obtained from the other pressure sensor positioned inside the patient 2900, which may then represent the patient's blood pressure, can be normalized. This allows calculations of the absolute blood pressure value of the patient 2900 can be done reliably while allowing compensation for any systematic errors (such as a change in the ambient air pressure around the patient 2900, for example when changing floors in a house, weather-based changes in air pressure or topographic altitude). This allows the patient's state of health to be determined very reliably in different environmental scenarios.

Additional details regarding the embodiment of the MCS system 100 and any related components and/or features described or shown in FIG. 29 are described in PCT Publication No. WO2020030706, filed Aug. 7, 2019, titled DEVICE AND METHOD FOR MONITORING THE STATE OF HEALTH OF A PATIENT, which is hereby incorporated by reference in its entirety.

FIGS. 30A and 30B show a front and a back view of an embodiment of the MCS controller 200. The MCS controller 200 may support operation of one or more cardiac or circulatory support systems, such as left ventricular support devices, ventricular assist devices, or MCS devices as described herein. The MCS controller 200 may include one more modules to provide power to the cardiac support systems. The MCS controller 200 may house electronic circuits to send and receive operational signals to the cardiac support system. The MCS controller 200 may house one or more hardware processors as described below to receive and process data, such as sensor data, from the cardiac support system. In some embodiments, the MCS controller 200 may have an integrated or self-contained design in which all or almost all of the components required for operation of the controller are housed within the controller. For example, any power supply components, such as transformers or AC/DC converters, may be housed within the MCS controller 200. As shown in FIG. 2, the MCS controller 200 may be wired to the pump (for example, the MCS device 100) via electronic wires extending through the catheter shaft 306 to the MCS device 100.

In some embodiments, the MCS controller 200 may include communications systems, or any other suitable systems, to allow the controller to be adapted to new or modified uses after construction of the MCS controller 200. For example, multiple modes of wired or wireless communication can be integrated within the MCS controller 200 to communicate with outside technology, such as, for example, RF, wifi, and/or Bluetooth. In some embodiments, the MCS controller 200 may have an RFID reader. In some embodiments, the MCS controller 200 may have systems or components that enable syncing patient data, telemedicine, patient monitoring, real time data collection, error reporting, and/or sharing maintenance records.

The MCS controller 200 may include a housing for these modules that support any of the cardiac support systems described herein. The housing may further include a handle 3002 to support portability. In contrast to conventional controllers such as Abiomed's Impella Controller, the MCS controller 200 disclosed herein may not include components required for purging. For example, the MCS controller 200 does not include a cassette for purging. The cassette typically delivers rinsing fluid to the catheter. However, the cassette requires significant real estate and makes the housing bigger and heavier. Due to the design improvements described herein, such as bearing design and sealed motor discussed herein, the MCS controller 200 does not include a cassette. Furthermore, in some embodiments, the controller 200 does not require a port for receiving a purging tube. Accordingly, the MCS controller 200 may be light and compact to support portability.

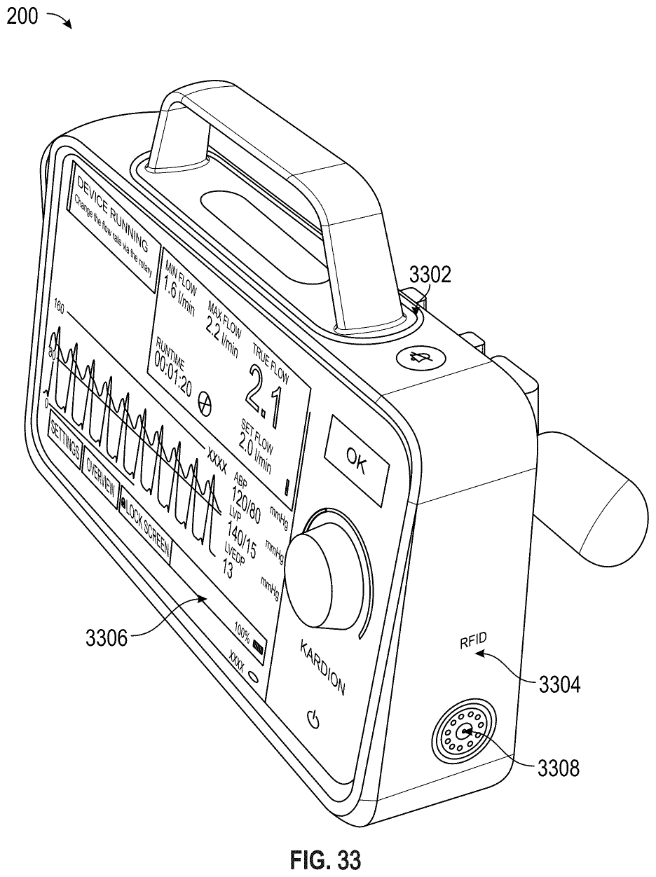

The MCS controller 200 may also include a cable management support 3004. In some embodiments, the cable management support 3004 is positioned on one end or side of the controller 200. The MCS controller 200 may also include a mount 3006 that may support mounting the MCS controller 200 to a pole in a clinical environment. The mount 3006 may rotate about an axis to support horizontal or vertical clamping. The mount 3006 may be rapidly locked into the desired orientation by quick fastening with a slipping clutch. In some instances, the mount 3006 is positioned away from the cable management support 3004. Furthermore, in some embodiments, the cable management support 3004 is positioned on a left end of the controller 200 as shown in FIG. 30A. The port 3308 (such as shown in FIG. 33) can be positioned on a side opposite from the cable management support 3004. In some instances, the control element 3008 discussed below is positioned on a side opposite from the cable management support 3004 and in close proximity to the port 3008. This may enable a user to have an improved interaction with the active components of the MCS controller 200. Therefore, the arrangement of all these elements in the MCS controller 200 as illustrated can improve operational experience and improve portability.

The MCS controller 200 can include a control element 3008. In some embodiment, the control element 3008 can provide haptic feedback. The control element 3008 can include a push button rotary dial. The control element 3008 can enable a user to change parameters on the MCS controller 200 to control one or more processes described herein. The control element 3008 may also include status indicator 3010 as illustrated in FIG. 30A. In some embodiments, the MCS controller 200 may include a separate confirmation control element. Furthermore, in some embodiments, aside from the separate confirmation control element, all the parameters can be modified using a single control element 3008. The grouping of controls in a dedicated area can improve user experience.

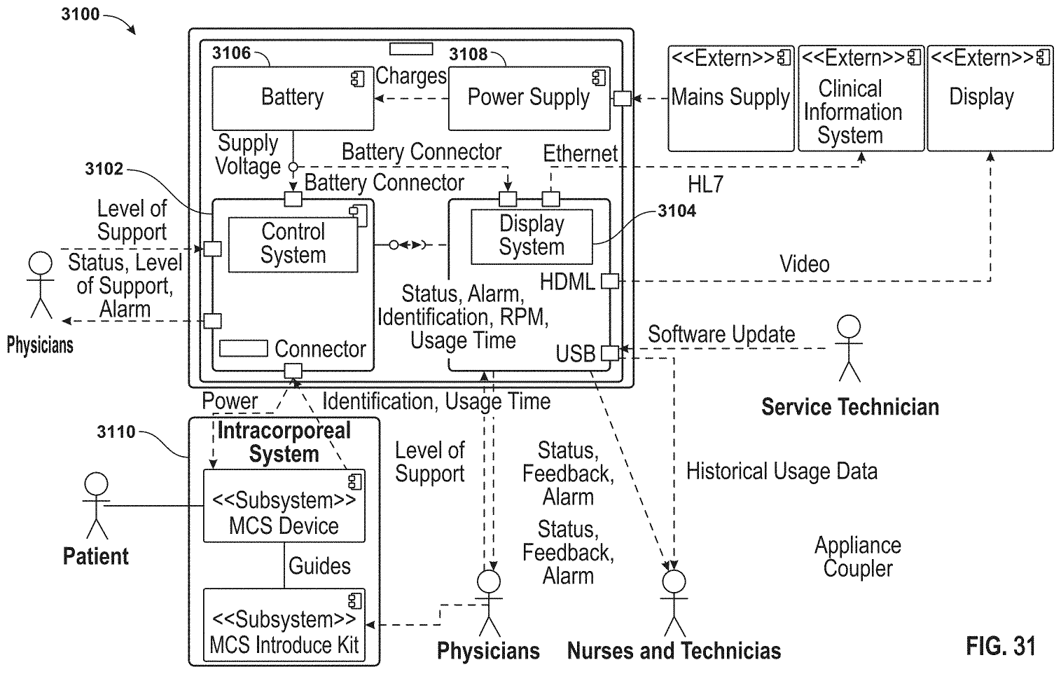

FIG. 31 illustrates a block diagram of an electronic system 3100 that can be included in the MCS controller 200. In some embodiments, the electronic system 3100 can include one or more circuit boards in conjunction with one or more hardware processors for controlling a MCS device 3110 (or the MCS device 100 as described herein). The electronic system 3100 can also receive signals, process signals, and transmit signals. The electronic system 3100 can further generate a display and/or alarms. The electronic system 3100 can include a control system 3102 and a display system 3104. In some embodiments, the display system 3104 can be integrated into the control system 3102 and is not separate as shown in FIG. 31. In some embodiments, it may be advantageous for the display system 3104 to be separate from the control system 3102. For example, in the event of failure of the control system 3102, the display system 3104 can serve as a backup.

The control system 3102 can include one or more hardware processors to control various aspects of the MCS device 3110. For example, the control system 3102 can control a motor of the MCS device 3110. The control system 3102 can also receive signals from the MCS device 3110 and process parameters. The parameters can include, for example, flow rate, motor current, ABP, LVP, LVEDP, etc. The control system 3102 can generate alarms and status of the MCS controller 200 and/or the MCS device 3110. In some embodiments, the control system 3102 can support multiple MCS devices 3110. The control system 3102 can transmit the generated alarms or status indicators to the display system 3104. The display system 3104 can include one or more hardware processors to receive processed data from the control system 3102 and render the processed data for display on a display screen. The control system 3102 can also include a memory for storing data.

The electronic system 3100 can also include a battery 3106 that can enable its electronics systems to operate without connection to an external power supply. The power supply interface 3108 can charge the battery 3106 from the external power supply. The control system 3102 can use the battery power to supply current to the motor of the MCS device 3110.

The one or more hardware processors can include microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein.

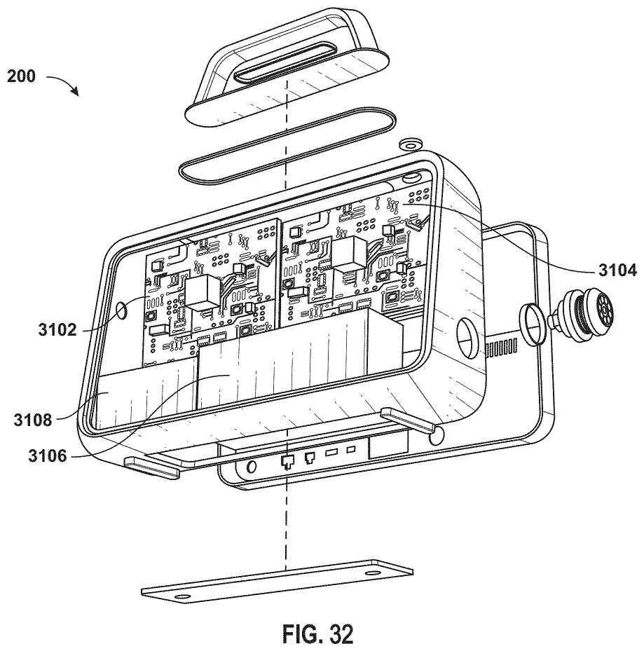

FIG. 32 is an exploded view of an embodiment of the MCS controller 200 having physical components corresponding to the features of the block diagram of the electronics system 3100 of FIG. 31. As shown in FIG. 32, the MCS controller 200 may include the control system 3102 and the display system 3104 including circuit boards arranged within the housing. The battery 3106 may be located within the bottom section of the housing. The power supply interface 3108 may be located within a corner of the housing.

FIG. 33 is a front perspective view of an embodiment of the MCS controller 200. In some embodiments, the MCS controller 200 can include an alarm feedback system, which can provide feedback to an operator regarding the operation of the MCS device 100 (or the MCS device 3110). In some embodiments, the alarm feedback system can be in the form of an LED 3302 as illustrated. The LED 3302 may be positioned so that it can be seen by an operator using the controller. As illustrated, the LED 3302 is positioned around the handle 3002. Therefore, it can be seen from positions 360° around the controller. The LED 3302 may be in the form of a ring (oval, oblong, circular, or any other suitable shape) wrapping the handle 3002. Such an LED 3302 may be visualized from any direction as long as the top of the controller is viewable. The control system 3302 can generate different colors or patterns for the LED 3302 to provide various alarms or status of the MCS controller 200 and/or the MCS device 100 (or MCS device 3100 as shown in FIG. 31).

The MCS controller 200 can further include a port 3308 that can receive a cable connected to an MCS device (for example, the MCS device 100). The port 3308 can support multiple versions of the MCS devices described herein. The MCS controller 200 can also include an RFID reader 3304 on a side of the MCS controller 200. The RFID reader 3304 can read badges of a sales representative and operate the device according to a particular demo mode. The MCS control 200 can include a glass cover 3306 that is tilted as shown in FIG. 33 to improve readability for the user.

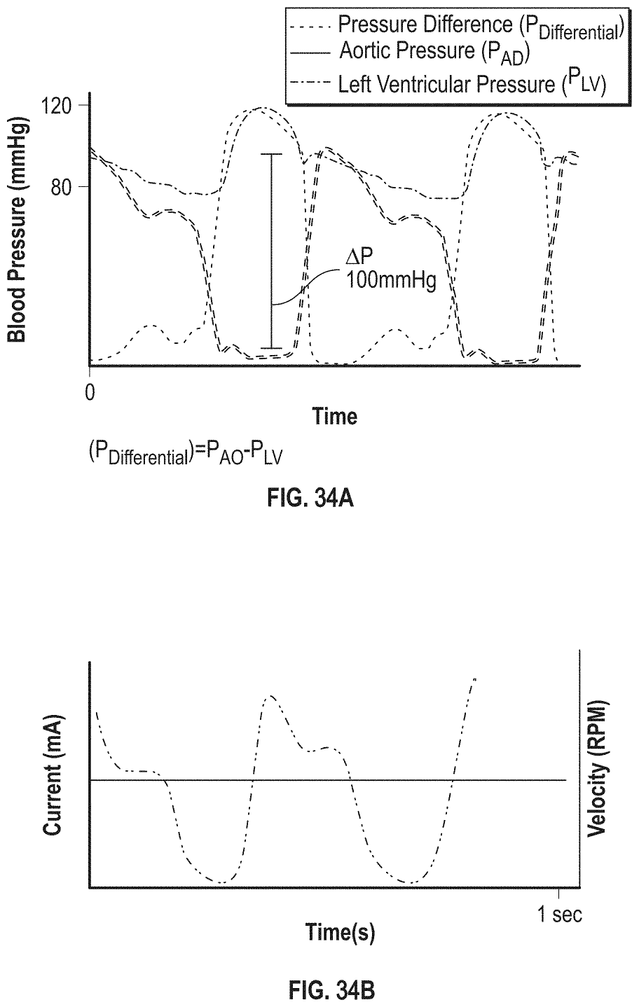

FIG. 34A illustrates a graph showing pressure differences between aortic pressure and left ventricular pressure, which may be typically pressure differences. In some instances, the MCS device 3110 (or the MCS device 100) can be positioned between the two locations of the heart corresponding to the different pressure levels (for example, left ventricle and aortic arch). Therefore, the MCS device 3110 may operate against a pressure difference shown in FIG. 34A. Accordingly, the motor of the MCS device 3110 may in some instances work with the pressure and in other instances against the pressure. Therefore, it was observed that to keep the velocity of the motor, for example, rotational speed of a motor shaft, constant or approximately stable, the current supplied to the motor would need to change based on the pressure differential.

FIG. 34B shows the applied current for a constant velocity (for example, constant motor velocity). The current curve of FIG. 34B follows a similar behavior as to the pressure differential curve of FIG. 34A. In some embodiments, the control system 3102 can control a motor to run at constant velocity by varying the motor current. The variation in the motor current can be used by the control system 3102 to probe the differential pressure, and therefore physiology of the patient, operating conditions, and machine conditions.

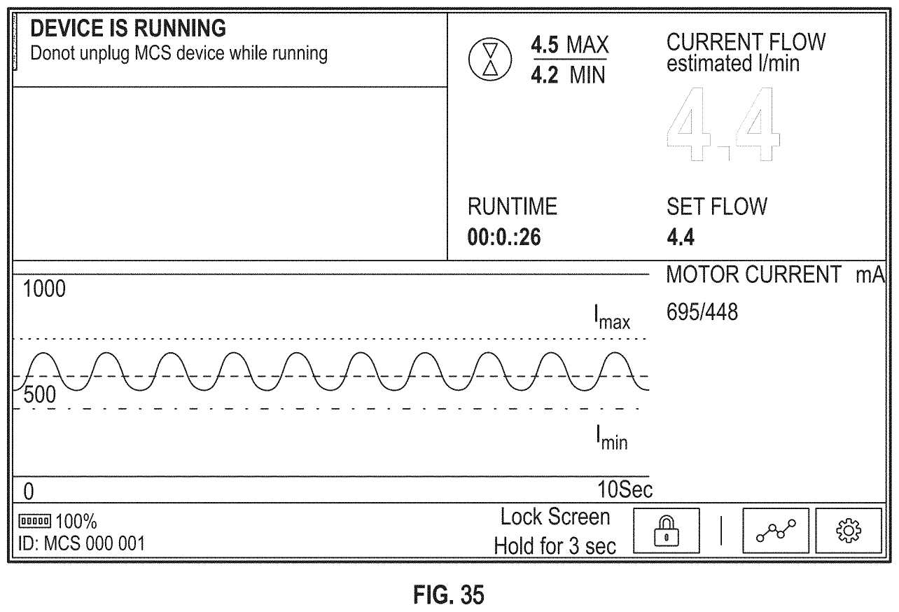

FIG. 35 illustrates an example user interface that can display flow rate parameters and motor current. The user interface can also display the parameters as a graph plotted with time. The user interface may be shown on the MCS controller 200, for example, on the display.

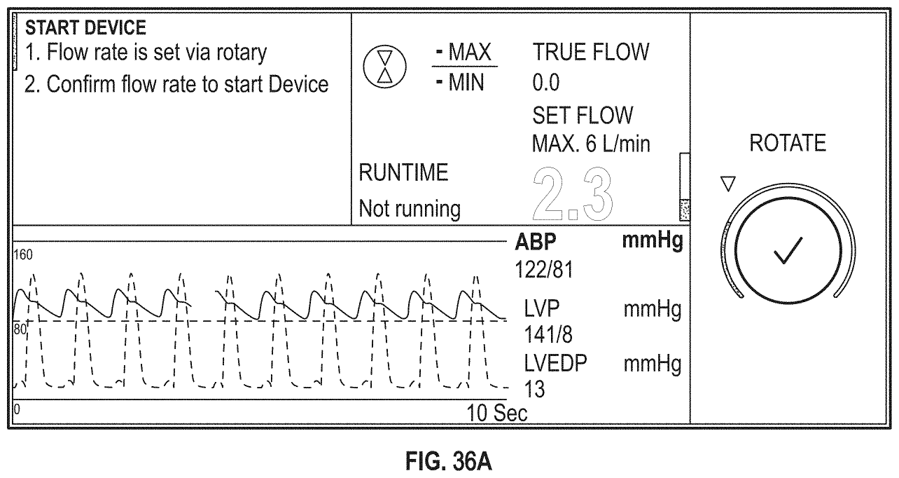

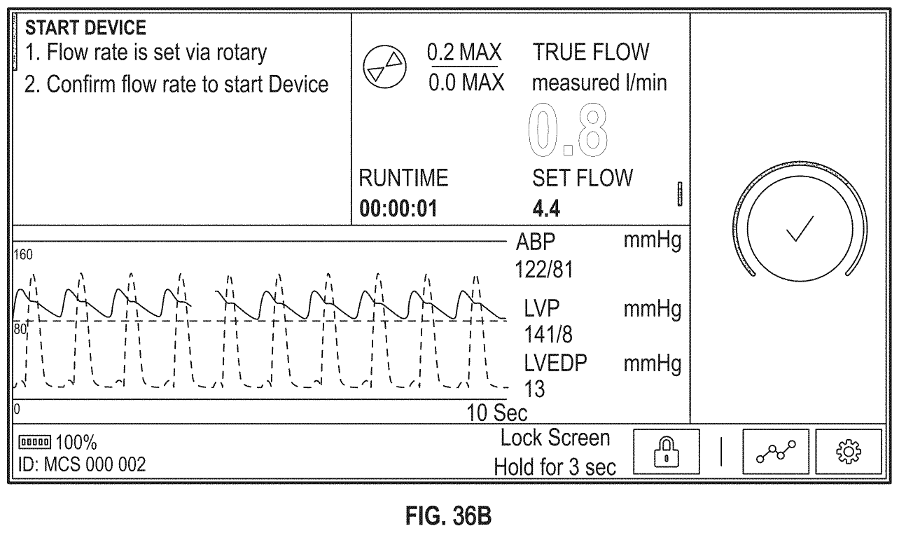

FIG. 36A illustrates an example user interface in a configuration mode where the control element 3008 can be used to modify parameters, such as setting the flow rate. The control element 3008 can include a visual feedback system directly on the knob and/or adjacent to the knob. FIG. 36B shows an example user interface during operation mode. Comparing FIGS. 36A and 36B, certain text on the user interface can be highlighted or emphasized depending on the modes. In the configuration mode, the set flow rate is enlarged. In operational mode, the flow rate is enlarged. This improves readability for the users particularly when the user interface includes several parameters.

In some embodiments, only some of the user interfaces may be available depending on the type of the MCS device 3110 (or the MCS device 100) connected with the MCS controller 200. For example, some devices discussed above may not include any sensors and may not support all the user interfaces discussed above. These sensor-less devices may be lower cost and smaller.

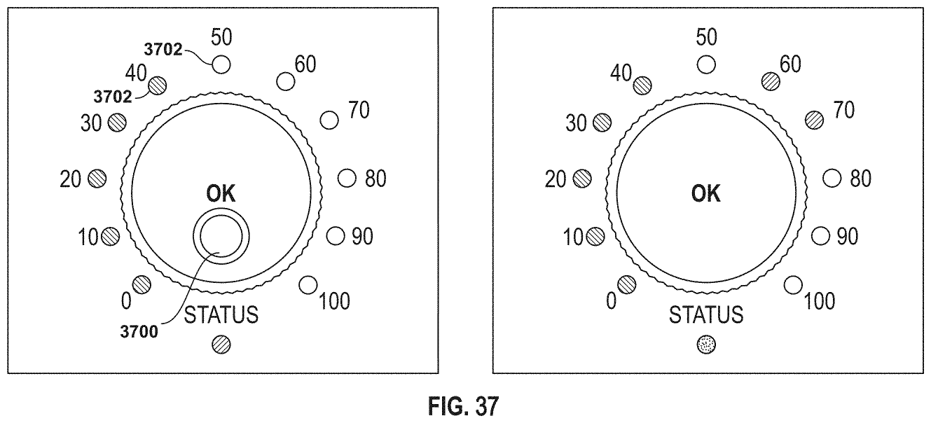

FIG. 37 illustrates an embodiment of an electronic control element 3700 and visual indicators 3702. The electronic control element 3700 can include a display on the face of the dial. Furthermore, the visual indicators 3702 can indicate status of the motor or other operating conditions as the dial is rotated.

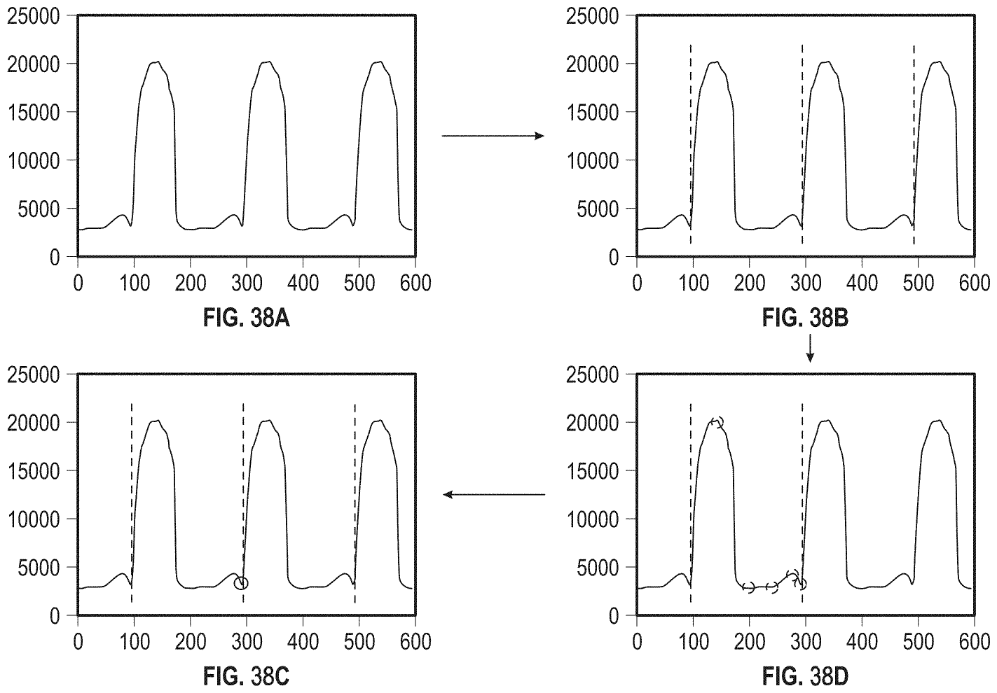

FIGS. 38A-38D are example left ventricle (LV) pressure curves illustrating a process for determining left ventricular end-diastolic pressure (LVEDP). The control system 3102 can document the status and operational parameters, which may be transferred to an EMR system via network communications.

The control system 3102 can measure LVEDP. FIGS. 38A-38D illustrate a series of steps for the determination of LVDEP from the measured LV pressure curve. FIG. 38A illustrates an example LV pressure curve measured with 100 MHz sampling rate. The control system 3102 can process the measured LV pressure curve to determine LVDEP. For example, the control system 3102 can identify a largest positive gradient in the LV curve as illustrated in FIG. 38B. This can identify the pulse value. Other techniques can be used to identify a start of a pulse. Once pulses are identified, the control system 3102 can find maxima and minima in the LV curve between two steep positive slopes as illustrated in FIG. 38B. This can also yield systolic and diastolic values. In some instances, the control system 3102 can identify a minimum value left of the 2^{nd} slope as illustrated in FIG. 38D. This value can represent the LVEDP determination.

As discussed above, for example with respect to FIG. 34B, controlling or synchronizing motor current with heart and measuring the motor current can enable the control system 3102 to probe the differential pressure through measuring current, and therefore physiological processes of the patient, operating conditions, and machine conditions. Physiological processes may include when the pump is hitting the wall of the heart. In some instances, the motor current is kept constant while measuring the change in RPM. In some instances, a separate flow or pressure sensor is not required to probe physiological processes. The motor design including a motor controller, such as the MCS controller 200, can enable high resolution current measurement. In some instances, motor controller is sensorless (for example, the motor controller may not include a Hall sensor). In some instances, the control system 3102 may operate the motor in a pulsatile mode to improve heart recovery.

Any embodiments of the MCS devices or systems, and features thereof, described herein may include various additional features or modifications, such as those described, for example, in PCT Pub. No. WO 2019/229223, filed on May 30, 2019, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, in U.S. patent application Ser. No. 17/057,252, filed Jun. 18, 2021, titled AXIAL-FLOW PUMP FOR A VENTRICULAR ASSIST DEVICE AND METHOD FOR PRODUCING AN AXIAL-FLOW PUMP FOR A VENTRICULAR ASSIST DEVICE, in PCT Pub. No. WO 2019/229214, filed on May 30, 2019, titled PUMP HOUSING DEVICE, METHOD FOR PRODUCING A PUMP HOUSING DEVICE, AND PUMP HAVING A PUMP HOUSING DEVICE, in U.S. patent application Ser. No. 17/057,548, filed May 19, 2021, titled PUMP HOUSING DEVICE, METHOD FOR PRODUCING A PUMP HOUSING DEVICE, AND PUMP HAVING A PUMP HOUSING DEVICE, in PCT Pub. No. WO 2020/01179, filed on Jul. 9, 2019, titled IMPELLER FOR AN IMPLANTABLE, VASCULAR SUPPORT SYSTEM, in U.S. patent application Ser. No. 17/258,853, filed Jul. 27, 2021, titled IMPELLER FOR AN IMPLANTABLE, VASCULAR SUPPORT SYSTEM, in PCT Pub. No. WO 2020/011797, filed on Jul. 9, 2019, titled IMPELLER HOUSING FOR AN IMPLANTABLE, VASCULAR SUPPORT SYSTEM, in U.S. patent application Ser. No. 17/258,861, filed Jul. 27, 2021, titled IMPELLER HOUSING FOR AN IMPLANTABLE, VASCULAR SUPPORT SYSTEM, in PCT Pub. No. WO 2020/030706, filed on Aug. 7, 2019, titled DEVICE AND METHOD FOR MONITORING THE STATE OF HEALTH OF A PATIENT, in U.S. patent application Ser. No. 17/266,056, filed Oct. 13, 2021, titled DEVICE AND METHOD FOR MONITORING THE STATE OF HEALTH OF A PATIENT, in PCT Pub. No. WO 2019/234149, filed on Jun. 6, 2019, titled SENSOR HEAD DEVICE FOR A MINIMAL INVASIVE VENTRICULAR ASSIST DEVICE AND METHOD FOR PRODUCING SUCH A SENSOR HEAD DEVICE, in U.S. patent application Ser. No. 15/734,036, filed Jun. 8, 2021, titled SENSOR HEAD DEVICE FOR A MINIMAL INVASIVE VENTRICULAR ASSIST DEVICE AND METHOD FOR PRODUCING SUCH A SENSOR HEAD DEVICE, in PCT Pub. No. WO 2019/234166, filed on Jun. 6, 2019, titled METHOD FOR DETERMINING A FLOW SPEED OF A FLUID FLOWING THROUGH AN IMPLANTED, VASCULAR ASSISTANCE SYSTEM AND IMPLANTABLE, VASCULAR ASSISTANCE SYSTEM, in U.S. patent application Ser. No. 15/734,523, filed Oct. 15, 2021, titled SYSTEMS AND METHODS FOR DETERMINING A FLOW SPEED OF A FLUID FLOWING THROUGH A CARDIAC ASSIST DEVICE, in PCT Pub. No. WO 2019/219874, filed May 16, 2019, titled PERMANENT-MAGNETIC RADIAL ROTATING JOINT AND MICROPUMP COMPRISING SUCH A RADIAL ROTATING JOINT, in U.S. application Ser. No. 17/055,059, filed Jun. 29, 2021, titled PERMANENT-MAGNETIC RADIAL ROTATING JOINT AND MICROPUMP COMPRISING SUCH A RADIAL ROTATING JOINT, in PCT Pub. No. WO 2020/030700, filed Aug. 7, 2019, titled BEARING DEVICE FOR A HEART SUPPORT SYSTEM, AND METHOD FOR RINSING A SPACE IN A BEARING DEVICE FOR A HEART SUPPORT SYSTEM, in U.S. application Ser. No. 17/266,044, filed Sep. 29, 2021, titled BEARING DEVICE FOR A HEART SUPPORT SYSTEM, AND METHOD FOR RINSING A SPACE IN A BEARING DEVICE FOR A HEART SUPPORT SYSTEM, in PCT Pub. No. WO 2019/234163, filed Jun. 6, 2019, titled METHOD AND SYSTEM FOR DETERMINING THE SPEED OF SOUND IN A FLUID IN THE REGION OF AN IMPLANTED VASCULAR SUPPORT SYSTEM, in U.S. patent application Ser. No. 15/734,322, filed Jun. 14, 2021, titled METHOD AND SYSTEM FOR DETERMINING THE SPEED OF SOUND IN A FLUID IN THE REGION OF AN IMPLANTED VASCULAR SUPPORT SYSTEM, in PCT Pub. No. WO 2019/234146, filed Jun. 6, 2019, titled LINE DEVICE FOR A VENTRICULAR ASSIST DEVICE AND METHOD FOR PRODUCING A LINE DEVICE, in U.S. patent application Ser. No. 15/734,331, filed Jun. 14, 2021, titled CONDUIT FOR A CARDIAC ASSIST DEVICE AND METHOD FOR PRODUCING A CONDUIT, in PCT Pub. No. WO 2019/234164, filed Jun. 6, 2019, titled METHOD FOR DETERMINING A FLOW RATE OF A FLUID FLOWING THROUGH AN IMPLANTED VASCULAR SUPPORT SYSTEM, AND IMPLANTABLE VASCULAR SUPPORT SYSTEM, in U.S. patent application Ser. No. 15/734,353, filed Jul. 16, 2021, titled METHOD FOR DETERMINING A FLOW RATE OF A FLUID FLOWING THROUGH AN IMPLANTED VASCULAR SUPPORT SYSTEM, AND IMPLANTABLE VASCULAR SUPPORT SYSTEM, in PCT Pub. No. WO 2019/234162, filed Jun. 6, 2019, titled METHOD FOR DETERMINING A FLUID TOTAL VOLUME FLOW IN THE REGION OF AN IMPLANTED VASCULAR SUPPORT SYSTEM AND IMPLANTABLE VASCULAR SUPPORT SYSTEM, in U.S. patent application Ser. No. 15/734,010, filed Jul. 9, 2021, titled METHOD FOR DETERMINING A FLUID TOTAL VOLUME FLOW IN THE REGION OF AN IMPLANTED VASCULAR SUPPORT SYSTEM AND IMPLANTABLE VASCULAR SUPPORT SYSTEM, in PCT Pub. No. WO 2019/064707, filed Sep. 24, 2019, titled METHOD AND SYSTEM FOR DETERMINING A FLOW SPEED OF A FLUID FLOWING THROUGH AN IMPLANTED, VASCULAR ASSISTANCE SYSTEM, in U.S. patent application Ser. No. 17/274,354, filed Mar. 8, 2021, titled METHOD AND SYSTEM FOR DETERMINING A FLOW SPEED OF A FLUID FLOWING THROUGH AN IMPLANTED, VASCULAR ASSISTANCE SYSTEM, in PCT Pub. No. WO 2019/229210, filed May 30, 2019, titled LINE DEVICE FOR CONDUCTING A BLOOD FLOW FOR A HEART SUPPORT SYSTEM, AND PRODUCTION AND ASSEMBLY METHOD, in U.S. patent application Ser. No. 17/057,355, filed May 18, 2021, titled LINE DEVICE FOR CONDUCTING A BLOOD FLOW FOR A HEART SUPPORT SYSTEM, AND PRODUCTION AND ASSEMBLY METHOD, each of which are hereby incorporated by reference herein in their entirety for all purposes and forms a part of this specification.

### EXAMPLE EMBODIMENTS

The following are numbered example embodiments of various embodiments of the mechanical circulatory support systems and methods disclosed herein. Any of the below Examples 1-34, or any other examples disclosed herein, may be combined in whole or in part. Elements of the examples disclosed herein are not limiting.

Example 1: A mechanical circulatory support system, comprising an elongate flexible catheter shaft, having a proximal end and a distal end, and a circulatory support device carried by the distal end of the shaft. The circulatory support device comprising a tubular housing, a motor having a shaft that is rotationally fixed with respect to a drive magnet array, an impeller rotationally fixed with respect to a driven magnet array, and a sealed motor housing coupled with the tubular housing, and encasing the motor and the drive magnet array.

Example 2: The mechanical circulatory support system or method of any of the Examples 1-34 or any other embodiment described herein, wherein the motor is configured to rotate the drive magnet array via the shaft, wherein the rotating drive magnet array magnetically communicates with the driven magnet array through the sealed motor housing to cause the impeller to rotate.

Example 3: The mechanical circulatory support system or method of any of the Examples 1-34 or any other embodiment described herein, wherein the driven magnet array and the drive magnet array at least partially axially overlap.

Example 4: The mechanical circulatory support system or method of any of the Examples 1-34 or any other embodiment described herein, wherein the driven magnet array is arranged axially staggered in relation to the drive magnet array.

Example 5: The mechanical circulatory support system or method of any of the Examples 1-34 or any other embodiment described herein, wherein the system does not require purging.

Example 6: The mechanical circulatory support system or method of any of the Examples 1-34 or any other embodiment described herein, further comprising a controller that does not include a purging component.

Example 7: The mechanical circulatory support system or method of any of the Examples 1-34 or any other embodiment described herein, wherein the controller does not include a cassette or a port for purging.

Example 8: The mechanical circulatory support system or method of any of the Examples 1-34 or any other embodiment described herein, further comprising an ultrasound sensor configured to detect blood volume flow using pulsed Doppler measurements.

Example 9: The mechanical circulatory support system or method of any of the Examples 1-34 or any other embodiment described herein, wherein the system is configured to detect the blood volume flow using an operating parameter of the mechanical circulatory support when a pulse repetition rate of the ultrasound sensor does not exceed twice a maximum Doppler frequency shift of the blood flow.

Example 10: The mechanical circulatory support system or method of any of the Examples 1-34 or any other embodiment described herein, wherein the operating parameter comprises a rotation rate of the drive magnet array or a differential pressure across the mechanical circulatory support.

Example 11: The mechanical circulatory support system or method of any of the Examples 1-34 or any other embodiment described herein, wherein the ultrasound sensor comprises an ultrasound transducer proximate a blood inlet port of the housing.

Example 12: The mechanical circulatory support system or method of any of the Examples 1-34 or any other embodiment described herein, further comprising a display device for monitoring a state of health of a patient, and a first pressure sensor and a second pressure sensor in communication with the display device to provide information related to a blood pressure difference, a pulse wave velocity of a blood pulse wave, and/or an elasticity of a blood vessel.

Example 13: The mechanical circulatory support system or method of any of the Examples 1-34 or any other embodiment described herein, further comprising a sensor head device at a distal end of the tubular housing, the sensor head device comprising a sensor carrying element comprising at least one sensor cavity configured to receive at least one sensor, and at least one signal transmitter cavity configured to receive at least one signal transmitter.

Example 14: The mechanical circulatory support system or method of any of the Examples 1-34 or any other embodiment described herein, further comprising one or more of the following arranged on an electrically conductive element: a temperature sensor, a pressure sensor, or a signal transmitter comprising an ultrasound element.

Example 15: The mechanical circulatory support system or method of any of the Examples 1-34 or any other embodiment described herein, wherein the driven magnet array comprises a Halbach array.

Example 16: The mechanical circulatory support system or method of any of the Examples 1-34 or any other embodiment described herein, wherein the drive magnet array comprises a magnetization being radial or parallel.

Example 17: The mechanical circulatory support system or method of any of the Examples 1-34 or any other embodiment described herein, wherein the drive and driven magnet arrays each comprise a same amount of pole pairs.

Example 18: The mechanical circulatory support system or method of any of the Examples 1-34 or any other embodiment described herein, further comprising an intermediate space between the sealed motor housing and the driven magnet array for guiding a flushing blood flow.

Example 19: The mechanical circulatory support system or method of any of the Examples 1-34 or any other embodiment described herein, wherein the impeller comprises at least one flushing outlet for discharging the flushing blood flow from the intermediate space.

Example 20: The mechanical circulatory support system or method of any of the Examples 1-34 or any other embodiment described herein, the tubular housing further comprising an inlet tube, and an electrical conducting element attached to the inlet tube, wherein the electrical conducting element comprises a plurality of layers and a sensor contact region configured to contact at least one sensor.

Example 21: The mechanical circulatory support system or method of any of the Examples 1-34 or any other embodiment described herein, the tubular housing further comprising an inlet tube arranged between a sensor head unit located at a distal end of the tubular housing and an end unit located proximal to the conduit, a first connecting element arranged between the inlet tube and the sensor head unit, and a second connecting element arranged between the inlet tube and the end unit.

Example 22: The mechanical circulatory support system or method of any of the Examples 1-34 or any other embodiment described herein, wherein a distal or proximal end of the tubular housing comprises an attachment section configured to attach to an adjacent component of the circulatory support device.

Example 23: The mechanical circulatory support system or method of any of the Examples 1-34 or any other embodiment described herein, wherein the attachment section is configured to attach to the adjacent component via form-locking or force-locking.

Example 24: The mechanical circulatory support system or method of any of the Examples 1-34 or any other embodiment described herein, further comprising a removable guidewire guide tube.

Example 25: The mechanical circulatory support system or method of any of the Examples 1-34 or any other embodiment described herein, wherein the guide tube enters a first guidewire port on a distal end of the tubular housing, exits the tubular housing via a second guidewire port on a side wall of the tubular housing distal to the impeller, reenters the tubular housing via a third guidewire port on a proximal side of the impeller, and extends proximally into the catheter shaft.

Example 26: The mechanical circulatory support system or method of any of the Examples 1-34 or any other embodiment described herein, further comprising at least one blood inlet port and at least one blood outlet port on the tubular housing separated by a flexible section of the tubular housing.

Example 27: The mechanical circulatory support system or method of any of the Examples 1-34 or any other embodiment described herein, wherein the tubular housing comprises an inlet tube coupled with an impeller cage.

Example 28: The mechanical circulatory support system or method of any of the Examples 1-34 or any other embodiment described herein, wherein the sealed motor housing is coupled with the tubular housing via the impeller cage.

Example 29: The mechanical circulatory support system or method of any of the Examples 1-34 or any other embodiment described herein, wherein the impeller cage at least partially encapsulates the sealed motor housing.

Example 30: The mechanical circulatory support system or method of any of the Examples 1-34 or any other embodiment described herein, wherein a distal end of the tubular housing comprises a nose piece having a sensor.

Example 31: A method of positioning a guidewire on a mechanical circulatory support device, the method comprising inserting a guidewire into a lumen of a catheter shaft coupled with the mechanical circulatory support device, the mechanical circulatory support device comprising an inlet tube, a pump impeller, a first guidewire port, and a second guidewire port, the first guidewire port being positioned proximal to the pump impeller and the second guidewire port being positioned distal to the pump impeller; extending the guidewire through the first guidewire port and towards the second guidewire port; and extending the guidewire through the second guidewire port, at least a portion of the guidewire distal from the second guidewire port is positioned inside the inlet tube, wherein at least a portion of the guidewire positioned distal from the first guidewire port and proximal from the second guidewire port is positioned on an outside surface of the inlet tube.

Example 32: A method of transcatheter delivery of a pump to the heart, the method comprising advancing the pump through vasculature, wherein the pump is advanced having a guidewire that extends through a first section of a catheter shaft located distal to the pump, through an interior of a tubular housing of the pump, through a sidewall of the tubular housing and external to the tubular housing, and into a second section of the catheter shaft located proximal to the pump.

Example 33: The mechanical circulatory support system or method of any of the Examples 1-34 or any other embodiment described herein, further comprising starting the motor and/or rotating the impeller prior to removal of the guidewire from the pump and/or prior to placement of the pump in the heart.

Example 34: The mechanical circulatory support system or method of any of the Examples 1-34 or any other embodiment described herein, further comprising leaving the guidewire in the pump during use of the pump so the guidewire and/or pump at least partially remains in the left ventricle.

### Terminology

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

What is claimed is:

1. A mechanical circulatory support system, comprising:
an elongate flexible catheter shaft, having a proximal end and a distal end;
a circulatory support device carried by the distal end of the shaft, the circulatory support device comprising:
a tubular housing;
a motor having a shaft that is rotationally fixed with respect to a drive magnet array;
an impeller, rotationally fixed with respect to a driven magnet array; and
a sealed motor housing coupled with the tubular housing, and encasing the motor and the drive magnet array.

2. The mechanical circulatory support system of claim 1, wherein the motor is configured to rotate the drive magnet array via the shaft, wherein the rotating drive magnet array magnetically communicates with the driven magnet array through the sealed motor housing to cause the impeller to rotate.

3. The mechanical circulatory support system of claim 1, wherein the driven magnet array and the drive magnet array at least partially axially overlap.

4. The mechanical circulatory support system of claim 1, wherein the driven magnet array is arranged axially staggered in relation to the drive magnet array.

5. The mechanical circulatory support system of claim 1, wherein the system does not require purging.

6. The mechanical circulatory support system of claim 1, further comprising a controller that does not include a purging component.

7. The mechanical circulatory support system of claim 6, wherein the controller does not include a cassette or a port for purging.

8. The mechanical circulatory support system of claim 1, further comprising an ultrasound sensor configured to detect blood volume flow using pulsed Doppler measurements.

9. The mechanical circulatory support system of claim 8, wherein the system is configured to detect the blood volume flow using an operating parameter of the circulatory support device when a pulse repetition rate of the ultrasound sensor does not exceed twice a maximum Doppler frequency shift of the blood volume flow.

10. The mechanical circulatory support system of claim 9, wherein the operating parameter comprises a rotation rate of the drive magnet array or a differential pressure across the circulatory support device.

11. The mechanical circulatory support system of claim 9, wherein the ultrasound sensor comprises an ultrasound transducer proximate a blood inlet port of the housing.

12. The mechanical circulatory support system of claim 1, further comprising:
a display device configured to display a state of health of a patient; and
a first pressure sensor and a second pressure sensor in communication with the display device to provide information related to a blood pressure difference, a pulse wave velocity of a blood pulse wave, and/or an elasticity of a blood vessel.

13. The mechanical circulatory support system of claim 1, further comprising a sensor head device at a distal end of the tubular housing, the sensor head device comprising:
a sensor carrying element comprising at least one sensor cavity configured to receive at least one sensor; and
at least one signal transmitter cavity configured to receive at least one signal transmitter.

14. The mechanical circulatory support system of claim 13, further comprising one or more of the following arranged on an electrical conductive element: a temperature sensor, a pressure sensor, and a signal transmitter comprising an ultrasound element.

15. The mechanical circulatory support system of claim 1, wherein the driven magnet array comprises a Halbach array.

16. The mechanical circulatory support system of claim 1, wherein the drive magnet array comprises a magnetization being radial or parallel.

17. The mechanical circulatory support system of claim 1, wherein the drive and driven magnet arrays each comprise a same amount of pole pairs.

18. The mechanical circulatory support system of claim 1, further comprising an intermediate space between the sealed motor housing and the driven magnet array configured to guide a flushing blood flow.

19. The mechanical circulatory support system of claim 18, wherein the impeller comprises at least one flushing outlet to discharge the flushing blood flow from the intermediate space.

20. The mechanical circulatory support system of claim 1, the tubular housing further comprising:
an inlet tube; and
an electrical conducting element attached to the inlet tube, wherein the electrical conducting element comprises a plurality of layers and a sensor contact region configured to contact at least one sensor.

21. The mechanical circulatory support system of claim 1, the tubular housing further comprising:
an inlet tube, arranged between a sensor head unit located at a distal end of the tubular housing and an end unit located proximal to the inlet tube;
a first connecting element arranged between the inlet tube and the sensor head unit; and
a second connecting element arranged between the inlet tube and the end unit.

22. The mechanical circulatory support system of claim 1, wherein a distal end or a proximal end of the tubular housing comprises an attachment section configured to attach to an adjacent component of the circulatory support device.

23. The mechanical circulatory support system of claim 22, wherein the attachment section is configured to attach to the adjacent component via form-locking or force-locking.

24. The mechanical circulatory support system of claim 1, further comprising a removable guidewire guide tube.

25. The mechanical circulatory support system of claim 24, wherein the guide tube enters a first guidewire port on a distal end of the tubular housing, exits the tubular housing via a second guidewire port on a side wall of the tubular housing distal to the impeller, reenters the tubular housing via a third guidewire port on a proximal side of the impeller, and extends proximally into the catheter shaft.

26. The mechanical circulatory support system of claim 1, further comprising at least one blood inlet port and at least one blood outlet port on the tubular housing separated by a flexible section of the tubular housing.

27. The mechanical circulatory support system of claim 1, wherein the tubular housing comprises an inlet tube coupled with an impeller cage.

28. The mechanical circulatory support system of claim 27, wherein the sealed motor housing is coupled with the tubular housing via the impeller cage.

29. The mechanical circulatory support system of claim 27, wherein the impeller cage at least partially encapsulates the sealed motor housing.

30. The mechanical circulatory support system of claim 1, wherein a distal end of the tubular housing comprises a nose piece having a sensor.

31. A method of positioning a guidewire on a mechanical circulatory support device, the method comprising:
inserting a guidewire into a lumen of a catheter shaft coupled with the mechanical circulatory support device, the mechanical circulatory support device comprising an inlet tube, a pump impeller, a first guidewire port, and a second guidewire port, the first guidewire port being positioned proximal to the pump impeller and the second guidewire port being positioned distal to the pump impeller;
extending the guidewire through the first guidewire port and towards the second guidewire port; and
extending the guidewire through the second guidewire port, at least a portion of the guidewire distal from the second guidewire port is positioned inside the inlet tube,
wherein at least a portion of the guidewire positioned distal from the first guidewire port and proximal from the second guidewire port is positioned on an outside surface of the inlet tube.

32. A method of transcatheter delivery of a pump to a heart, the method comprising:
advancing the pump through vasculature, wherein the pump is advanced having a guidewire that extends through a first section of a catheter shaft located distal to the pump, through an interior of a tubular housing of the pump, through a sidewall of the tubular housing and external to the tubular housing, and into a second section of the catheter shaft located proximal to the pump.

33. The method of claim 32, further comprising starting the motor and/or rotating the impeller prior to removal of the guidewire from the pump and/or prior to placement of the pump in the heart.

34. The method of claim 32, further comprising leaving the guidewire in the pump during use of the pump so the guidewire and/or pump at least partially remains in the left ventricle.
